(19) United States
(12) Patent Application Publication
(10) Pub. No.: US 2021/0163453 A1
(43) Pub. Date: Jun. 3, 2021

(54) DERIVATIVES OF PAPAVERINE THAT ARE EFFECTIVE HYPOXIC TUMOR RADIOSENSITIZERS

(71) Applicant: Ohio State Innovation Foundation, Columbus, OH (US)

(72) Inventors: Nicholas DENKO, Dublin, OH (US); Ioanna PAPANDREOU, Columbus, OH (US); Martin BENEJ, Upper Arlington, OH (US); Ching-Shih CHEN, Taichung (TW); Sandip VIBHUTE, Columbus, OH (US)

(21) Appl. No.: 17/262,989

(22) PCT Filed: Jul. 23, 2019
(86) PCT No.: PCT/US2019/043082

§ 371 Date: Jan. 25, 2021

Related U.S. Application Data
(60) Provisional application No. 62/702,201, filed on Jul. 23, 2018.

(51) Int. Cl. C07D 405/06; C07D 217/24; C07D 239/72; C07D 401/06; A61P 35/00

(52) U.S. Cl. CPC C07D 405/06; C07D 217/24; A61P 35/00; C07D 401/06; C07D 239/72

(57) ABSTRACT

Disclosed herein are compounds, compositions, and methods for inhibiting mitochondrial oxygen consumption in a cancerous tissue. The compounds, compositions, and methods can be used to treat a subject with hypoxic cancerous tissue.

### CROSS-REFERENCE TO RELATED APPLICATIONS

This application claims benefit of U.S. Provisional Application No. 62/702,201, filed Jul. 23, 2018, which is hereby incorporated herein by reference in its entirety.

### STATEMENT REGARDING FEDERALLY SPONSORED RESEARCH OR DEVELOPMENT

This invention was made with government support under Grant No. P01 CA067166 20 awarded by the National Cancer Institute. The government has certain rights in the invention.

### BACKGROUND OF THE DISCLOSURE

Hypoxia is a common microenvironmental feature of solid tumors (Brown, J. M. et al. Cancer Res 58, 1408-1416 (1998)) that exists because the supply of oxygen is insufficient to meet the metabolic demand of the tumor (Epstein, T. et al. Cancer Metab 2, 7 (2014) and Semenza, G. L. et al. The Journal of clinical investigation 123, 3664-3671 (2013)). The poorly formed tumor blood vessels make it difficult to therapeutically increase oxygen delivery to reduce hypoxia (Harrison, D. K. et al. Adv Exp Med Biol 812, 25-31 (2014)). It has been recognized for over 60 years that hypoxia protects organisms from the detrimental effects of ionizing radiation. The “oxygen enhancement ratio” or OER is approximately two and a half- to three-fold. This means it takes two and a half- to three-times the dose of radiation delivered to very hypoxic cells to get the same amount of cell kill if those cells had been fully oxygenated. Biophysical analysis supports a model where oxygen acts as an electrophile to fix DNA damage within nanoseconds of radiation delivery, lack of fixation allows for resolution of metastable DNA radical intermediates in hypoxia.

Even though tumor hypoxia has been studied preclinically, clinical approaches designed to overcome hypoxia have disappointing results. Strategies designed to deliver more oxygen to the tumor, deliver oxygen-mimetics, or deliver drugs with preferential toxicity towards hypoxic cells have all been effective in rodent studies but failed in human trials. The most widely studied strategy has been to increase delivery of oxygen to tumors. However, this is limited by the inherent nature of the poorly formed and poorly functioning tumor vascular tree. Vessels in transplanted and spontaneous tumors have blind ends, breaks, and tortuous paths, all of which reduce laminar flow and decrease oxygen delivery.

There is a need for compounds and methods that can treat hypoxic tumors. The compounds, compositions, and methods disclosed herein address these and other needs.

### SUMMARY OF THE DISCLOSURE

Disclosed herein are compounds represented by a structure having the Formula I

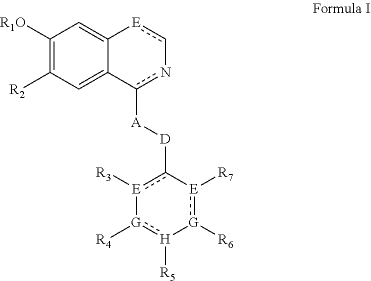

wherein A and D are independently present or absent and are independently selected from CR′R″, NR′, and O, wherein R′ and R″ are independently for each occurrence selected from hydrogen, hydroxyl, halogen, amine, alkylamine, C₁-C₆ alkyl, C₁-C₆ alkyl halide, C₁-C₆ alkoxy, or R′ and R″ combine together with the atom to which they are attached form a carbonyl group; E, G, and H are independently selected from C, N, O, and S; R¹ and R² are independently selected from hydrogen, C₁-C₆ alkyl, C₁-C₆ alkyl halide, C₁-C₆ alkoxy, and C₁-C₆ alkylamine; R³ to R⁷ are independently selected from hydrogen, hydroxyl, C₁-C₆ alkyl, C₁-C₆ alkyl halide, C₁-C₆ alkoxy, and C₁-C₆ alkylamine or R³ and R⁴ or R⁴ and R⁵ or R⁵ and R⁶ or R⁶ and R⁷ combine together with the atoms to which they are attached form a C₅-C₈ aryl or heteroaryl, or C₅-C₈ cycloalkenyl or heterocycloalkenyl; wherein R³ to R⁷ are independently unsubstituted or substituted with hydroxyl, halogen, C₁-C₃ alkyl, C₁-C₃ alkenyl, or C₁-C₃ alkyl halide; and ----- represents a bond and is independently for each occurrence absent or present, wherein when A is CH₂ and D is absent, then R¹, R², R⁴, and R⁵ are not simultaneously OMe or when A is CH₂, D is absent, R¹ and R² are OMe, then R⁴ and R⁵ do not combine to form and unsubstituted aryl.

In some embodiments of Formula I, the compounds can be represented by a structure having the Formula I′ as described herein, wherein E is selected from C and N.

In some embodiments of Formula I and I′, A is present and D is absent. In some embodiments, of Formula I and I′, both A and D are present. In some embodiments, of Formula I and I′, both A and D are absent.

In some embodiments of Formula I and I′, A can be selected from CR′R″ and O. In some embodiments of Formula I and I′, D can be selected from CR′R″ and O. In some embodiments of Formula I and I′, A and D can both be CR′R″. In some embodiments of Formula I and I′, A can be CR′R″ and D can be O.

In some embodiments of Formula I and I′, R′ and R″ can be independently for each occurrence selected from hydrogen, hydroxyl, C₁-C₆ alkyl, or R′ and R″ combine together with the atom to which they are attached form a carbonyl. For example, R′ and R″ can be hydrogen. In some examples, R′ is hydrogen and at least one occurrence of R″ is hydroxyl. In other examples, at least one occurrence of R′ and R″ combine together with the atom to which they are attached form a carbonyl.

In some embodiments of Formula I and I′, R¹ can be selected from a C₁-C₆ alkyl. For example, R¹ can be selected from a C₁-C₂ alkyl.

In some embodiments of Formula I and I′, R² can be independently selected from hydrogen or a C₁-C₆ alkoxy. For example, R² can be selected from a C₁-C₂ alkoxy. In some examples, R² can be hydrogen.

In some embodiments of Formula I and I′, R³, R⁴, R, R⁶, and R⁷ can be independently selected from hydrogen, hydroxyl, C₁-C₆ alkyl, C₁-C₆ alkyl halide, C₁-C₆ alkoxy, or R³ and R⁴ or R⁴ and R⁵ combine together with the atoms to which they are attached form a C₅-C₈ aryl or heteroaryl, or C₅—C cycloalkenyl or heterocycloalkenyl, wherein R³ to R⁷ are independently unsubstituted or substituted with hydroxyl, halogen, C₁-C₃ alkyl, C₁-C₃ alkenyl, or C₁-C₃ alkyl halide. For example, R³ and R⁷ are hydrogen. In some examples, R⁴, R⁵, and R⁶ can be independently selected from hydrogen, hydroxyl, C₁-C₆ alkyl, C₁-C₆ alkyl halide, or C₁-C₆ alkoxy. In other examples, R³ and R⁴ or R⁴ and R combine together with the atoms to which they are attached form a C₆ aryl, a C₆ heteroaryl, or a C₅ heterocycloalkenyl.

Pharmaceutical composition or formulation comprising a compound disclosed herein are also disclosed. In some examples, the pharmaceutical compositions comprises papaverine.

Methods for inhibiting mitochondrial oxygen consumption in a cancerous tissue within a subject, the method comprising administering to the subject a pharmaceutical composition disclosed herein are also disclosed. The methods can further comprise irradiating the cancerous tissue with an ionizing radiation for an effective period. The method can cause a therapeutic injury resulting in the reduction of at least one of surface area, the depth, and the amount of the tissue affected by the cancerous condition. Such cancerous tissue can be selected from the group consisting of colorectal cancer, breast cancer, bladder cancer, brain cancer, cervical cancer, gastrointestinal cancer, genitourinary cancer, head and neck cancer, lung cancer, pancreatic cancer, prostate cancer, renal cancer, skin cancer, and testicular cancer.

The compositions can be administered within 30 minutes to 4 hours, preferably within 30 minutes to 90 minutes of administering the ionizing radiation. In some examples, the cancerous tissue can be irradiated with at least 1 fraction, preferably from 1 to 30 fractions of radiation per day, the total fraction of radiation being from about 25 to about 75 Gray.

The method can further comprise administering a chemotherapeutic drug.

### BRIEF DESCRIPTION OF THE DRAWINGS

FIGS. 1A-1E show papaverine reduces OCR by inhibition of mitochondrial complex I. FIG. 1A shows representative Seahorse data showing the effect of PPV injected at time A on OCR in EO771 cells. FIG. 1B shows competition assay with papaverine and rotenone in EO771 cells. FIG. 1C shows succinate rescue assay in permeabilized EO771 cells. PPV or rotenone were injected alone or in combination with complex III inhibitor antimycin A (AntA) at time A, succinate was injected at time B. FIG. 1D shows dose response analysis in A549 cell line. FIG. 1E shows drug washout experiment in A549 cells, the % OCR response to 3 h of drug treatment (pink) and removal 2 hours (blue) and 1 hour (black) prior to OCR measurement. Error bars represent standard deviation.

FIGS. 2A-2D are OCR measurements and cell viability assessments. FIG. 2A-2B are representative of OCR measurement in competition assay between PPV and complex I inhibitors piericidin A (FIG. 2A) and capsaicin (FIG. 2B). Values represent the means+−SD. FIGS. 2C-2D show trypan blue cell viability assessment (of n=3) of A549 cells treated with 100 μM papaverine or 1 μM rotenone in normoxia (FIG. 2C) and hypoxia (FIG. 2D) (T=72 h). Bar charts represent mean of viable cells in duplicates+−standard deviation.

FIGS. 3A-3G show papaverine reduces tumor hypoxia and enhances response to radiation therapy. FIG. 3A shows FD-NIRS analysis of baseline oxygenation levels in immune-deficient mice with A549 or EO771 flank xenografts (n=5). P values were calculated against thigh muscle with two-tailed two-sample t test. FIG. 3B-3C show normalized tissue oxygenation in EO771 (b) or A549 (c) tumor-bearing mice after injection of 2 mg/kg PPV or vehicle saline (readings taken every minute, and curve is average of 5 traces). P value was analyzed by linear mixed model with autoregressive correlation structure at T=30-40 minutes. FIG. 3D shows representative immunofluorescence analysis image showing hypoxic marker pimonidazole (green) and Hoechst nuclear counterstain (blue) in tumor cryosections from EO771 tumor-bearing mice treated with saline or 2 mg per kg body weight PPV. FIG. 3E shows quantification of hypoxic fractions in tumor cryosections in d (n=4). Values are mean area covered by pimonidazole-positive cells evaluated from 20 images per animal+−SEM. P value was calculated with two-tailed two-sample t test. FIG. 3F shows quantification of tumor growth delay of orthotopic EO771 tumors grown in nude mice receiving either 2 mg/kg PPV (magenta), 5 Gy XRT (blue) or 2 mg/kg PPV 35 minutes before 5 Gy XRT (red) (n=9-10). Curves represent mean tumor volumes+−SEM. P values were calculated against XRT with two-tailed two-sample t test.

FIG. 3G shows quantification of tumor growth of heterotopic A549 flank xenografts in nude mice receiving either 8 Gy XRT (magenta), 2 mg/kg PPV 35 minutes after (blue) or prior to (red) 8 Gy XRT (n=6). Curves represent mean tumor volumes+−SEM. P values were calculated against XRT with two-tailed two-sample t test. *P<0.05; **P<0.01; ***P<0.001; ****P<0.0001; n.s., not significant.

FIGS. 4A-4G show tumor growth before and after radiosensitization with PPV. FIG. 4A-4B show in vitro radiation survival assessment of A549 cell line treated with 10 μm PPV in normoxia (FIG. 4A) and anoxia (FIG. 4B). Normoxic cells were irradiated in triplicates on a cell culture dish, anoxic cells were irradiated in high density suspension after sealing with mineral oil 45 minutes before radiation. Values represent averaged colony counts+−standard deviation. FIG. 4C show quantification of tumor growth delay in orthotopic EO771 tumors gown in C₅₇B16 mice receiving either 5 Gy XRT (magenta) or 2 mg/kg PPV 35 minutes prior to 5 Gy XRT (blue) (n=3-4). Values are mean tumor volumes+−SEM. FIG. 4D show quantification of tumor growth delay of heterotopic EO771 flank tumors in nude mice receiving either 5 Gy XRT (magenta) or 2 mg/kg PPV 35 minutes after (blue) or prior to 5 Gy XRT (red) (n=4). Values are mean tumor volumes+−SEM. FIG. 4E-4F show tumor growth delay. When tumors reached 4-fold volume increase, groups receiving no treatment or PPV only (FIG. 4E); and XRT only or PPV followed by XRT (FIG. 4F), tumor weights were compared. Values are mean tumor weights+−SEM (n=8). P values were calculated with two-tailed two-sample t test. FIG. 4G are related to FIG. 4E-4F tumor growth delay. Representative images of orthotopic EO771 tumors harvested on day 11 after treatment with XRT (top) or 2 mg/kg PPV followed by XRT (bottom). *P<0.05; **P<0.01; ***P<0.001; ****P<0.0001; n.s., not significant.

FIGS. 5A-5E show papaverine radiosensitizes through complex I inhibition. FIG. 5A shows Western blot of NDI1 expression in mitochondrial fractions of parent A549 and NDUFV1 KO cells. FIG. 5B shows representative trypan blue viability assay (n=3 experiments) of cells grown in galactose-only media (T=96 h). Values represent mean viable cells+−SD. FIG. 5C shows seahorse analysis showing response of parent A549 and NDUFV1 KO+−NDI1 cells to 10 μM papaverine or 1 μM rotenone. Values are mean+−SD. FIG. 5D shows quantification of hypoxic fractions in tumor cryosections from NDUFV1 KO NDI1 flank tumor-bearing mice treated with 2 mg/kg PPV or vehicle (n=3). Value is mean area covered by pimonidazole-positive cells evaluated from 20 images per animal+−standard error of the mean. P value was calculated with two-tailed two-sample t test. FIG. 5E shows quantification of tumor growth delay of heterotopic NDUFV1 KO NDI1 flank xenografts receiving either 8 Gy XRT (magenta) or 2 mg/kg PPV 35 minutes prior to 8 Gy XRT (blue) (n=4). Curves represent mean tumor volumes+−SEM. P values were calculated against XRT with two-tailed two-sample t test. *P<0.05; **P<0.01; ***P<0.001; ****P<0.0001; n.s., not significant.

FIGS. 6A-6E show uncropped western blot membrane used for FIG. 5A showing NDUFV1 and PDH E1α levels (FIG. 6A) before probing for NDI1 (FIG. 6B). FIG. 6C shows quantification of baseline OCR in parent A549 and NDUFV1 KO+−NDI1 cells. Values represent mean OCR from at least 5 replicates+−SD. FIG. 6D shows in vitro radiation survival assessment of A549 NDUFV1 KO cells+−NDI1 treated with 10 m PPV. Curves represent averaged colony counts+−SD. FIG. 6E shows tumor growth delay of heterotopic NDUFV1 KO NDI1 (different NDUFV1 KO clone) flank xenografts in nude mice after receiving either 8 Gy dose of XRT (magenta) or 2 mg per kg body weight PPV 35 minutes prior to 8 Gy XRT (blue) (n=4). Values are mean tumor volumes+−SEM. P values were calculated against XRT with two-tailed two-sample t test. *P<0.05; **P<0.01; ***P<0.001; ****P<0.0001; n.s., not significant.

FIGS. 7A-7D show OCR and cellular toxicity data. FIG. 7A shows seahorse OCR data showing no effect of increasing concentrations of phosphodiesterase 5 inhibitor sildenafil citrate (0, 5, 10, 20 M) in Panc1 cell line. Values are means+−SD. FIG. 7B shows seahorse OCR analysis showing no effect of treatment with increasing concentrations of synthetic cAMP analog 8-Bromo-cAMP (0, 10, 20 M) in Panc1 cells. Values are means+−SD. FIG. 7C shows quantification of OCR and PDE10 inhibitory activities of 41 novel PPV derivatives normalized to PPV evaluated by Seahorse (OCR inhibition at 10 μM concentration) and PDE10A enzymatic assay (PDE10A inhibition at 1 μM). Bars represent mean OCR and PDE10 inhibitory activities from at least 5 (OCR) or 3 (PDE) replicates. All experiments were repeated at least twice. FIG. 7D shows cellular toxicity of PPV derivatives in vitro measured by trypan blue after 72 h treatment at 10 uM. Values are mean+−SEM.

FIGS. 8A-8F show papaverine can be re-engineered to remove PDE activity. FIG. 8A shows the structures of papaverine and the lead derivatives SMV-23 and SMV-32. FIG. 8B shows calculation of OCR IC50 in A549 cells by Seahorse, n=5 replicates per group. FIG. 8C shows calculation of PDE10A IC20 by PDE10A2 enzymatic assay, n=3 replicates per group. FIG. 8D shows acute toxicity in wild-type C57BL/6 mice (n=3). Anesthetized mice were treated with a rapid 6 mg/kg dose of either PPV, SMV-23 or SMV-32 by tail vein injection and observed for 1 hour. FIG. 8E shows quantification of hypoxic fractions in tumor cryosections from orthotopic EO771 tumor-bearing immune-competent mice treated with 2 mg per kg body weight PPV, SMV-23, SMV-32 or vehicle (n=3). Bar graphs represent the mean area covered by pimonidazole-positive cells evaluated from 20 images per animal+−standard error of the mean. P value was calculated against control with two-tailed two-sample t test. FIG. 8F shows tumor growth delay of heterotopic EO771 flank tumors in nude mice receiving either 5 Gy XRT (red) or 2 mg/kg SMV-23 (blue), or SMV-32 (magenta) or PPV (gray) 35 minutes prior to 5 Gy XRT (n=8). Mean volumes+−SEM. P values were calculated against XRT with two-tailed two-sample t test. *P<0.05; **P<0.01; ***P<0.001; ****P<0.0001; n.s., not significant.

FIG. 9 shows seahorse XF dose response of PPV as an inhibitor of oxygen consumption (OCR) in murine EO771 breast cancer cells in real time. PPV injected at T=20 min is effective at low micromolar concentrations.

FIGS. 10A-10B show PPV is a complex 1 inhibitor. FIG. 10A shows seahorse OCR analysis of PPV given first (A) shows it can interfere with the subsequent ability of rotenone to inhibit OCR (B). FIG. 10B shows after OCR inhibition by PPV or rotenone, addition of complex 2 substrate succinate can rescue OCR, but not if antimycin A is added (complex 3 inhibitor).

FIGS. 11A-11B show oxygenation traces from subcutaneous EO771 (FIG. 11A) or A549 (FIG. 11B) tumors grown in nude mice. Data plotted is the average oxygenation in 5 tumors or normal tissue. 4 measures per minute were averaged and only PPV effect on tumor oxygenation is statistically significant P<0.01

FIG. 12 shows papaverine is not an inherent radiosensitizer. A549 cells in suspension were treated with 5 μM PPV for one hour prior to radiation and plating in fresh media for colony formation post radiation. (N=2 experiments performed in triplicate).

FIGS. 13A-13B show PPV enhances tumor growth delay. FIG. 13A shows addition of PPV prior to radiation of orthotopic breast cancers significantly adds to tumor growth delay after 5Gy (n=3/group). FIG. 13B shows addition of PPV before, but not after XRT enhances tumor growth delay in flank A549 tumors. Increase in tumor growth delay greater than 3-fold when compared to XRT alone. (n=5 tumors per group+/−SEM, experiment was concluded when tumors reached 3× initial volume).

FIGS. 14A-14E summarize results obtained using ODD-luciferase as an in vivo oxygen reporter. FIG. 14A shows in vitro RLU from ODDluc expressing MP2 cells in hypoxia or normoxia. FIG. 14B shows ODDluc tumors imaged with animals breathing carbogen or medical air for 30 min. FIG. 14C shows mouse BLI response to PPV or saline in ODDluc tumors (n=3-4, P<0.05). FIG. 14D shows the response of orthotopic MP2 CMVluc tumors to PPV or saline (n=3-4, P=NS). FIG. 14E shows the response of orthotopic MP2 ODDLuc tumors to SMV32 or saline. (n=3-4, P<0.05).

### DETAILED DESCRIPTION

### General Definitions

Terms used herein will have their customary meaning in the art unless specified otherwise. The organic moieties mentioned when defining variable positions within the general formulae described herein (e.g., the term “halogen”) are collective terms for the individual substituents encompassed by the organic moiety. The prefix Cn-Cm indicates in each case the possible number of carbon atoms in the group.

Throughout the description and claims of this specification the word “comprise” and other forms of the word, such as “comprising” and “comprises,” means including but not limited to, and is not intended to exclude, for example, other additives, components, integers, or steps.

As used in the description and the appended claims, the singular forms “a,” “an,” and “the” include plural referents unless the context clearly dictates otherwise. Thus, for example, reference to “a compound” includes mixtures of two or more such compounds, reference to “an additional chemotherapy agent” includes mixtures of two or more such agents, reference to “the composition” includes mixtures of two or more of such compositions, and the like.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed.

As used herein, by a “subject” is meant an individual. Thus, the “subject” can include domesticated animals (e.g., cats, dogs, etc.), livestock (e.g., cattle, horses, pigs, sheep, goats, etc.), laboratory animals (e.g., mouse, rabbit, rat, guinea pig, etc.), and birds. “Subject” can also include a mammal, such as a primate or a human.

By “reduce” or other forms of the word, such as “reducing” or “reduction,” is meant lowering of an event or characteristic. It is understood that this is typically in relation to some standard or expected value, in other words it is relative, but that it is not always necessary for the standard or relative value to be referred to. For example, “reduce oxygen consumption in the tumor cells” can refer to a decrease in the amount of oxygen consumed relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

The terms “treatment,” “treat,” “treating,” and grammatical variations thereof, are used interchangeably herein to refer to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

An “effective amount” of a compound or composition disclosed herein is that amount which is necessary to carry out the compound's or composition's function of ameliorating, diminishing, reversing, treating or preventing a condition, disease or disorder. Such amelioration only requires a reduction or alteration, not necessarily elimination.

### Chemical Definitions

The term “alkyl,” as used herein, refers to saturated straight, branched, cyclic, primary, secondary or tertiary hydrocarbons, including those having 1 to 20 atoms. In some embodiments, alkyl groups will include C₁-C₁₂, C₁-C₁₀, C₁-C₈, C₁-C₆, C₁-C₅, C₁-C₄, C₁-C₃, C₁-C₂, or C₁ alkyl groups. Examples of C₁-C₁₀ alkyl groups include, but are not limited to, methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, heptyl, octyl, 2-ethylhexyl, nonyl and decyl groups, as well as their isomers. Examples of C₁-C₄-alkyl groups include, for example, methyl, ethyl, propyl, 1-methylethyl, butyl, 1-methylpropyl, 2-methylpropyl and 1,1-dimethylethyl groups.

Cyclic alkyl groups or “cycloalkyl” groups, which are encompassed alkyl, include cycloalkyl groups having from 3 to 10 carbon atoms. Cycloalkyl groups can include a single ring, or multiple condensed rings. In some embodiments, cycloalkyl groups include C₃-C₄, C₄-C₇, C₅-C₇, C₄-C₆, or C₅-C₆ cyclic alkyl groups. Non-limiting examples of cycloalkyl groups include adamantyl, cyclopropyl, cyclobutyl, cyclopentyl, cyclohexyl, cycloheptyl, cyclooctyl and the like.

Alkyl groups can be unsubstituted or substituted with one or more moieties selected from the group consisting of alkyl, halo, haloalkyl, hydroxyl, carboxyl, acyl, acyloxy, amino, alkyl- or dialkylamino, amido, arylamino, alkoxy, aryloxy, nitro, cyano, azido, thiol, imino, sulfonic acid, sulfate, sulfonyl, sulfanyl, sulfinyl, sulfamonyl, ester, phosphonyl, phosphinyl, phosphoryl, phosphine, thioester, thioether, acid halide, anhydride, oxime, hydrazine, carbamate, phosphoric acid, phosphate, phosphonate, or any other viable functional group that does not inhibit the biological activity of the compounds of the invention, either unprotected, or protected as necessary, as known to those skilled in the art, for example, as described in Greene, et al., Protective Groups in Organic Synthesis, John Wiley and Sons, Third Edition, 1999, hereby incorporated by reference.

Terms including the term “alkyl,” such as “alkylcycloalkyl,” “cycloalkylalkyl,” “alkylamino,” or “dialkylamino,” will be understood to comprise an alkyl group as defined above linked to another functional group, where the group is linked to the compound through the last group listed, as understood by those of skill in the art.

The term “alkenyl,” as used herein, refers to both straight and branched carbon chains which have at least one carbon-carbon double bond. In some embodiments, alkenyl groups can include C₂-C₂₀ alkenyl groups. In other embodiments, alkenyl can include C₂-C₁₂, C₂-C₁₀, C₂-C₈, C₂-C₆ or C₂-C₄ alkenyl groups. In one embodiment of alkenyl, the number of double bonds is 1-3, in another embodiment of alkenyl, the number of double bonds is one or two. Other ranges of carbon-carbon double bonds and carbon numbers are also contemplated depending on the location of the alkenyl moiety on the molecule. “C₂-C₁₀-alkenyl” groups may include more than one double bond in the chain. The one or more unsaturations within the alkenyl group may be located at any position(s) within the carbon chain as valence permits. In some embodiments, when the alkenyl group is covalently bound to one or more additional moieties, the carbon atom(s) in the alkenyl group that are covalently bound to the one or more additional moieties are not part of a carbon-carbon double bond within the alkenyl group. Examples of alkenyl groups include, but are not limited to, ethenyl, 1-propenyl, 2-propenyl, 1-methyl-ethenyl, 1-butenyl, 2-butenyl, 3-butenyl, 1-methyl-1-propenyl, 2-methyl-1-propenyl, 1-methyl-2-propenyl, 2-methyl-2-propenyl; 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl groups.

The term “alkynyl,” as used herein, refers to both straight and branched carbon chains which have at least one carbon-carbon triple bond. In one embodiment of alkynyl, the number of triple bonds is 1-3; in another embodiment of alkynyl, the number of triple bonds is one or two. In some embodiments, alkynyl groups include from C₂-C₂₀ alkynyl groups. In other embodiments, alkynyl groups may include C₂-C₁₂, C₂-C₁₀, C₂-C₈, C₂-C₆ or C₂-C₄ alkynyl groups. Other ranges of carbon-carbon triple bonds and carbon numbers are also contemplated depending on the location of the alkenyl moiety on the molecule. For example, the term “C₂-C₁₀-alkynyl” as used herein refers to a straight-chain or branched unsaturated hydrocarbon group having 2 to 10 carbon atoms and containing at least one triple bond, such as ethynyl, prop-1-yn-1-yl, prop-2-yn-1-yl, n-but-1-yn-1-yl, n-but-1-yn-3-yl, n-but-1-yn-4-yl, n-but-2-yn-1-yl, n-pent-1-yn-1-yl, n-pent-1-yn-3-yl, n-pent-1-yn-4-yl, n-pent-1-yn-5-yl, n-pent-2-yn-1-yl, n-pent-2-yn-4-yl, n-pent-2-yn-5-yl, 3-methylbut-1-yn-3-yl, 3-methylbut-1-yn-4-yl, n-hex-1-yn-1-yl, n-hex-1-yn-3-yl, n-hex-1-yn-4-yl, n-hex-1-yn-5-yl, n-hex-1-yn-6-yl, n-hex-2-yn-1-yl, n-hex-2-yn-4-yl, n-hex-2-yn-5-yl, n-hex-2-yn-6-yl, n-hex-3-yn-1-yl, n-hex-3-yn-2-yl, 3-methylpent-1-yn-1-yl, 3-methylpent-1-yn-3-yl, 3-methylpent-1-yn-4-yl, 3-methylpent-1-yn-5-yl, 4-methylpent-1-yn-1-yl, 4-methylpent-2-yn-4-yl, and 4-methylpent-2-yn-5-yl groups.

The term “haloalkyl” or “alkylhalide,” as used herein refers to an alkyl group, as defined above, which is substituted by one or more halogen atoms. In some instances, the haloalkyl group can be an alkyl group substituted by one or more fluorine atoms. In certain instances, the haloalkyl group can be a perfluorinated alkyl group. For example C₁-C₄-haloalkyl includes, but is not limited to, chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, and pentafluoroethyl.

The term “alkoxy,” as used herein, refers to alkyl-O—, wherein alkyl refers to an alkyl group, as defined above. Similarly, the terms “alkenyloxy,” “alkynyloxy,” “haloalkoxy,” “haloalkenyloxy,” “haloalkynyloxy,” “cycloalkoxy,” “cycloalkenyloxy,” “halocycloalkoxy,” and “halocycloalkenyloxy” refer to the groups alkenyl-O—, alkynyl-O—, haloalkyl-O—, haloalkenyl-O—, haloalkynyl-O—, cycloalkyl-O—, cycloalkenyl-O—, halocycloalkyl-O—, and halocycloalkenyl-O—, respectively, wherein alkenyl, alkynyl, haloalkyl, haloalkenyl, haloalkynyl, cycloalkyl, cycloalkenyl, halocycloalkyl, and halocycloalkenyl are as defined above. Examples of C₁-C₆-alkoxy include, but are not limited to, methoxy, ethoxy, C₂H₅—CH₂O—, (CH₃)₂CHO—, n-butoxy, C₂H₅—CH(CH₃)O—, (CH₃)₂CH—CH₂O—, (CH₃)₃CO—, n-pentoxy, 1-methylbutoxy, 2-methylbutoxy, 3-methylbutoxy, 1,1-dimethylpropoxy, 1,2-dimethylpropoxy, 2,2-dimethyl-propoxy, 1-ethylpropoxy, n-hexoxy, 1-methylpentoxy, 2-methylpentoxy, 3-methylpentoxy, 4-methylpentoxy, 1,1-dimethylbutoxy, 1,2-dimethylbutoxy, 1,3-dimethylbutoxy, 2,2-dimethylbutoxy, 2,3-dimethylbutoxy, 3,3-dimethylbutoxy, 1-ethylbutoxy, 2-ethylbutoxy, 1,1,2-trimethylpropoxy, 1,2,2-trimethylpropoxy, 1-ethyl-1-methylpropoxy, and 1-ethyl-2-methylpropoxy.

The terms “alkylamino” and “dialkylamino,” as used herein, refer to alkyl-NH— and (alkyl)₂N— groups, where alkyl is as defined above. Similarly, the terms “haloalkylamino” and “halodialkylamino” refer to haloalkyl-NH— and (haloalkyl)₂-NH—, where haloalkyl is as defined above.

The term “aryl,” as used herein, refers to a monovalent aromatic carbocyclic group of from 6 to 14 carbon atoms. Aryl groups can include a single ring or multiple condensed rings. In some embodiments, aryl groups include C₆-C₁₀ aryl groups. Aryl groups include, but are not limited to, phenyl, biphenyl, naphthyl, tetrahydronaphtyl, phenylcyclopropyl and indanyl. Aryl groups may be unsubstituted or substituted by one or more moieties selected from halogen, cyano, nitro, hydroxy, mercapto, amino, alkyl, alkenyl, alkynyl, cycloalkyl, cycloalkenyl, haloalkyl, haloalkenyl, haloalkynyl, halocycloalkyl, halocycloalkenyl, alkoxy, alkenyloxy, alkynyloxy, haloalkoxy, haloalkenyloxy, haloalkynyloxy, cycloalkoxy, cycloalkenyloxy, halocycloalkoxy, halocycloalkenyloxy, alkylthio, haloalkylthio, cycloalkylthio, halocycloalkylthio, alkylsulfinyl, alkenylsulfinyl, alkynyl-sulfinyl, haloalkylsulfinyl, haloalkenylsulfinyl, haloalkynylsulfinyl, alkylsulfonyl, alkenylsulfonyl, alkynylsulfonyl, haloalkyl-sulfonyl, haloalkenylsulfonyl, haloalkynylsulfonyl, alkylamino, alkenylamino, alkynylamino, di(alkyl)amino, di(alkenyl)-amino, di(alkynyl)amino, or trialkylsilyl.

The term “alkylaryl,” as used herein, refers to an aryl group that is bonded to a parent compound through a diradical alkylene bridge, (—CH₂-)ₙ, where n is 1-12 and where “aryl” is as defined above.

The term “alkylcycloalkyl,” as used herein, refers to a cycloalkyl group that is bonded to a parent compound through a diradical alkylene bridge, (—CH₂-)ₙ, where n is 1-12 and where “cycloalkyl” is as defined above. The term “cycloalkylalkyl,” as used herein, refers to a cycloalkyl group, as defined above, which is substituted by an alkyl group, as defined above.

The term “heteroaryl,” as used herein, refers to a monovalent aromatic group of from 1 to 15 carbon atoms (e.g., from 1 to 10 carbon atoms, from 2 to 8 carbon atoms, from 3 to 6 carbon atoms, or from 4 to 6 carbon atoms) having one or more heteroatoms within the ring. The heteroaryl group can include from 1 to 4 heteroatoms, from 1 to 3 heteroatoms, or from 1 to 2 heteroatoms. In some cases, the heteroatom(s) incorporated into the ring are oxygen, nitrogen, sulfur, or combinations thereof. When present, the nitrogen and sulfur heteroatoms may optionally be oxidized. Heteroaryl groups can have a single ring (e.g., pyridyl or furyl) or multiple condensed rings provided that the point of attachment is through a heteroaryl ring atom. Preferred heteroaryls include pyridyl, pyridazinyl, pyrimidinyl, pyrazinyl, triazinyl, pyrrolyl, indolyl, quinolinyl, isoquinolinyl, quinazolinyl, quinoxalinyl, furanyl, thiophenyl, furyl, pyrrolyl, imidazolyl, oxazolyl, isoxazolyl, isothiazolyl, pyrazolyl, benzofuranyl, and benzothiophenyl. Heteroaryl rings may be unsubstituted or substituted by one or more moieties as described for aryl above.

The term “alkylheteroaryl,” as used herein, refers to a heteroaryl group that is bonded to a parent compound through a diradical alkylene bridge, (—CH₂-)ₙ, where n is 1-12 and where “heteroaryl” is as defined above.

The terms “cycloheteroalkyl,” “heterocyclyl,” “heterocyclic,” and “heterocyclo” are used herein interchangeably, and refer to fully saturated or unsaturated, cyclic groups, for example, 3 to 7 membered monocyclic or 4 to 7 membered monocyclic; 7 to 11 membered bicyclic, or 10 to 15 membered tricyclic ring systems, having one or more heteroatoms within the ring. The heterocyclyl group can include from 1 to 4 heteroatoms, from 1 to 3 heteroatoms, or from 1 to 2 heteroatoms. In some cases, the heteroatom(s) incorporated into the ring are oxygen, nitrogen, sulfur, or combinations thereof. When present, the nitrogen and sulfur heteroatoms may optionally be oxidized, and the nitrogen heteroatoms may optionally be quaternized. The heterocyclyl group may be attached at any heteroatom or carbon atom of the ring or ring system and may be unsubstituted or substituted by one or more moieties as described for aryl groups above.

Exemplary monocyclic heterocyclic groups include, but are not limited to, pyrrolidinyl, pyrrolyl, pyrazolyl, oxetanyl, pyrazolinyl, imidazolyl, imidazolinyl, imidazolidinyl, oxazolyl, oxazolidinyl, isoxazolinyl, isoxazolyl, thiazolyl, thiadiazolyl, thiazolidinyl, isothiazolyl, isothiazolidinyl, furyl, tetrahydrofuryl, thienyl, oxadiazolyl, piperidinyl, piperazinyl, 2-oxopiperazinyl, 2-oxopiperidinyl, 2-oxopyrrolodinyl, 2-oxoazepinyl, azepinyl, 4-piperidonyl, pyridinyl, pyrazinyl, pyrimidinyl, pyridazinyl, tetrahydropyranyl, morpholinyl, thiamorpholinyl, thiamorpholinyl sulfoxide, thiamorpholinyl sulfone, 1,3-dioxolane and tetrahydro-1,1-dioxothienyl, triazolyl, triazinyl, and the like.

The term “alkylheterocyclyl” and “alkylcycloheteroalkyl” are used herein interchangeably, and refer to a heterocyclyl group that is bonded to a parent compound through a diradical alkylene bridge, (—CH₂-)ₙ, where n is 1-12 and where “heterocyclyl” is as defined above. The term “heterocyclylalkyl,” as used herein, refers to a heterocyclyl group, as defined above, which is substituted by an alkyl group, as defined above.

The term “halogen,” as used herein, refers to the atoms fluorine, chlorine, bromine and iodine. The prefix halo- (e.g., as illustrated by the term haloalkyl) refers to all degrees of halogen substitution, from a single substitution to a perhalo substitution (e.g., as illustrated with methyl as chloromethyl (—CH₂Cl), dichloromethyl (—CHCl₂), trichloromethyl (—CCl₃)).

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc.

Compounds

Disclosed herein are compounds and compositions for inhibiting mitochondrial oxygen consumption in a cancerous tissue. In certain embodiments, the compounds and compositions can treat hypoxic tumors in a subject. The compounds preferably comprise a planar portion as well as a flexible linkage.

In some aspects of the present disclosure, compounds represented by a structure having the Formula I are disclosed:

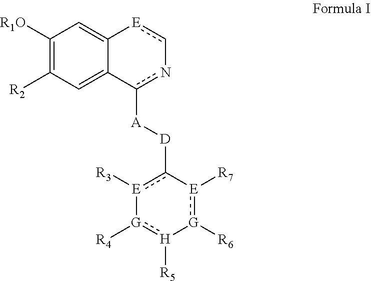

wherein
A and D can be independently present or absent and are independently selected from CR′R″, NR′, and O, wherein R′ and R″ are independently for each occurrence selected from hydrogen, hydroxyl, halogen, amine, alkylamine, C₁-C₆ alkyl, C₁-C₆ alkyl halide, C₁-C₆ alkoxy, or R′ and R″ combine together with the atom to which they are attached form a carbonyl group;
E, G, and H can be independently selected from C, N, O, and S;
R¹ and R² can be independently selected from hydrogen, C₁-C₆ alkyl, C₁-C₆ alkyl halide, C₁-C₆ alkoxy, and C₁-C₆ alkylamine;
R³ to R⁷ can be independently selected from hydrogen, hydroxyl, C₁-C₆ alkyl, C₁-C₆ alkyl halide, C₁-C₆ alkoxy, and C₁-C₆ alkylamine or R³ and R⁴ or R⁴ and R⁵ or R⁵ and R⁶ or R⁶ and R⁷ combine together with the atoms to which they are attached form a C₅-C₈ aryl or heteroaryl, or C₅-C₈ cycloalkenyl or heterocycloalkenyl; wherein R³ to R⁷ are independently unsubstituted or substituted with hydroxyl, halogen, C₁-C₃ alkyl, C₁-C₃ alkenyl, or C₁-C₃ alkyl halide; and
----- represents a bond and is independently for each occurrence absent or present.

In certain embodiments of Formula I, when A is CH₂ and D is absent, then R¹, R², R⁴, and R⁵ are not simultaneously OMe. In certain other embodiments of Formula I, when A is CH₂, D is absent, R¹ and R² are OMe, then R⁴ and R⁵ do not combine to form and unsubstituted aryl. In further embodiments of Formula I, R⁴ and R⁵ or R⁵ and R⁶ are not simultaneously OMe. In still further embodiments of Formula I, when A is CH₂ and D is absent, then R¹ and R² are not simultaneously OMe. For example, in some embodiments of Formula I, the compound is not papaverine or a

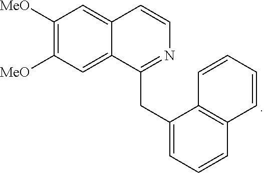 [SMILES: COc1cc2ccnc(Cc3cccc4ccccc34)c2cc1OC]

In some embodiments of Formula I, the compound can be represented by a structure having the Formula I′:

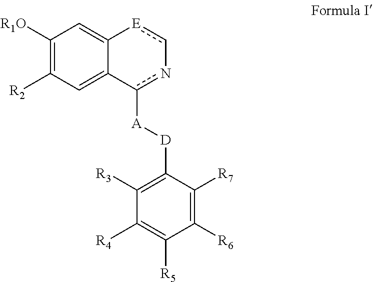

wherein A, D, R, R, and R¹ to R⁷ are as defined in Formula I.

For example, in some embodiments of Formula I′:
A and D are independently present or absent and are independently selected from CR′R″, NR′, and O, wherein R′ and R″ are independently for each occurrence selected from hydrogen, hydroxyl, halogen, amine, alkylamine, C₁-C₆ alkyl, C₁-C₆ alkyl halide, C₁-C₆ alkoxy, or R′ and R″ combine together with the atom to which they are attached form a carbonyl group;
R¹ and R² are independently selected from hydrogen, C₁-C₆ alkyl, C₁-C₆ alkyl halide, C₁-C₆ alkoxy, and C₁-C₆ alkylamine;
R³ to R⁷ are independently selected from hydrogen, hydroxyl, C₁-C₆ alkyl, C₁-C₆ alkyl halide, C₁-C₆ alkoxy, and C₁-C₆ alkylamine or R³ and R⁴ or R⁴ and R⁵ or R⁵ and R⁶ or R⁶ and R⁷ combine together with the atoms to which they are attached form a C₅-C₈ aryl or heteroaryl, or C₅-C₈ cycloalkenyl or heterocycloalkenyl; wherein R³ to R⁷ are independently unsubstituted or substituted with hydroxyl, halogen, C₁-C₃ alkyl, C₁-C₃ alkenyl, or C₁-C₃ alkyl halide; and
----- represents a bond and is independently for each occurrence absent or present,
wherein R⁴ and R⁵ or R⁵ and R⁶ are not simultaneously OMe, or when A is CH₂ and D is absent, then R¹ and R² are not simultaneously OMe.

In certain embodiments of Formula I, the compound can be represented by a structure having the Formula I-A to I-C:

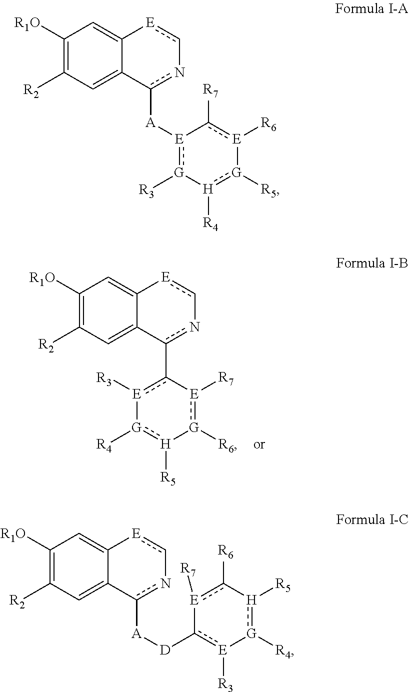

wherein
A is present in Formula I-A,
A and D are present in Formula I-C, and
A, D, E, G, H, R′, R″, and R¹ to R⁷ are as defined in Formula I.

For example, in some embodiments of Formula I-A to I-C:
A and D can be independently present or absent and are independently selected from CR′R″, NR′, and O, wherein R′ and R″ are independently for each occurrence selected from hydrogen, hydroxyl, halogen, amine, alkylamine, C₁-C₆ alkyl, C₁-C₆ alkyl halide, C₁-C₆ alkoxy, or R′ and R″ combine together with the atom to which they are attached form a carbonyl group;
E, G, and H can be independently selected from C, N, O, and S;
R¹ and R² can be independently selected from hydrogen, C₁-C₆ alkyl, C₁-C₆ alkyl halide, C₁-C₆ alkoxy, and C₁-C₆ alkylamine;
R³ to R⁷ can be independently selected from hydrogen, hydroxyl, C₁-C₆ alkyl, C₁-C₆ alkyl halide, C₁-C₆ alkoxy, and C₁-C₆ alkylamine or R³ and R⁴ or R⁴ and R⁵ or R⁵ and R⁶ or R⁶ and R⁷ combine together with the atoms to which they are attached form a C₅-C₈ aryl or heteroaryl, or C₅-C₈ cycloalkenyl or heterocycloalkenyl; wherein R³ to R⁷ are independently unsubstituted or substituted with hydroxyl, halogen, C₁-C₃ alkyl, C₁-C₃ alkenyl, or C₁-C₃ alkyl halide; and
----- represents a bond and is independently for each occurrence absent or present.

In certain embodiments of Formula I, the compound can be represented by a structure having the Formula I′-A to I′-C:

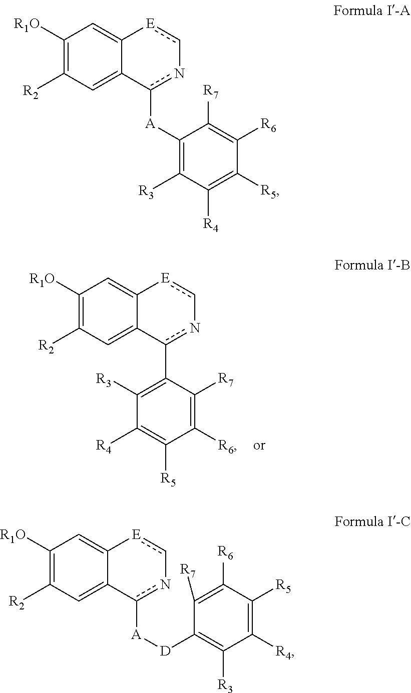

wherein
A is present in Formula I′-A,
A and D are present in Formula I′-C, and
A, D, R′, R″, and R¹ to R⁷ are as defined in Formula I.

For example, in some embodiments of Formula I′-A to I′-C:
A and D are independently present or absent and are independently selected from CR′R″, NR′, and O, wherein R′ and R″ are independently for each occurrence selected from hydrogen, hydroxyl, halogen, amine, alkylamine, C₁-C₆ alkyl, C₁-C₆ alkyl halide, C₁-C₆ alkoxy, or R′ and R″ combine together with the atom to which they are attached form a carbonyl group;
R¹ and R² are independently selected from hydrogen, C₁-C₆ alkyl, C₁-C₆ alkyl halide, C₁-C₆ alkoxy, and C₁-C₆ alkylamine;
R³ to R⁷ are independently selected from hydrogen, hydroxyl, C₁-C₆ alkyl, C₁-C₆ alkyl halide, C₁-C₆ alkoxy, and C₁-C₆ alkylamine or R³ and R⁴ or R⁴ and R⁵ or R⁵ and R⁶ or R⁶ and R⁷ combine together with the atoms to which they are attached form a C₅-C₈ aryl or heteroaryl, or C₅-C₈ cycloalkenyl or heterocycloalkenyl; wherein R³ to R⁷ are independently unsubstituted or substituted with hydroxyl, halogen, C₁-C₃ alkyl, C₁-C₃ alkenyl, or C₁-C₃ alkyl halide; and
----- represents a bond and is independently for each occurrence absent or present,
wherein R⁴ and R⁵ or R⁵ and R⁶ are not simultaneously OMe, or when A is CH₂ and D is absent, then R¹ and R² are not simultaneously OMe.

In certain embodiments of Formula I, the compound can be represented by a structure having the Formula I-A-1:

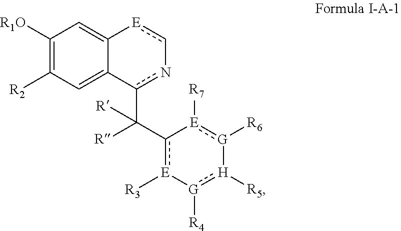

wherein
R′ and R″ are independently selected from hydrogen, hydroxyl, halogen, amine, alkylamine, C₁-C₆ alkyl, C₁-C₆ alkyl halide, C₁-C₆ alkoxy, or R′ and R″ combine together with the atom to which they are attached form a carbonyl;
R¹ is selected from hydrogen and C₁-C₆ alkyl;
R² is selected from hydrogen, hydroxyl, C₁-C₆ alkyl, and C₁-C₆ alkoxy; and
R³ to R⁷ are independently selected from hydrogen, hydroxyl, C₁-C₆ alkyl, C₁-C₆ alkyl halide, C₁-C₆ alkoxy, and C₁-C₆ alkylamine or R³ and R⁴ or R⁴ and R⁵ or R⁵ and R⁶ or R⁶ and R⁷ combine together with the atoms to which they are attached form a C₅-C₈ aryl or heteroaryl, or C₅—C cycloalkenyl or heterocycloalkenyl,
wherein R³ to R⁷ are independently unsubstituted or substituted with hydroxyl, halogen, C₁-C₃ alkyl, C₁-C₃ alkenyl, or C₁-C₃ alkyl halide.

In certain embodiments of Formula I, the compound can be represented by a structure having the Formula I′-A-1:

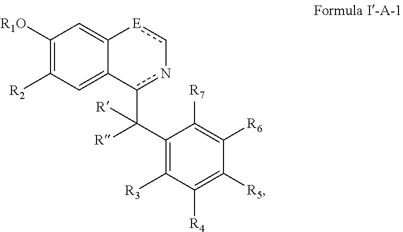

wherein
R′ and R″ are independently selected from hydrogen, hydroxyl, halogen, amine, alkylamine, C₁-C₆ alkyl, C₁-C₆ alkyl halide, C₁-C₆ alkoxy, or R′ and R″ combine together with the atom to which they are attached form a carbonyl;
R¹ is selected from hydrogen and C₁-C₆ alkyl;
R² is selected from hydrogen, hydroxyl, C₁-C₆ alkyl, and C₁-C₆ alkoxy; and
R³ to R⁷ are independently selected from hydrogen, hydroxyl, C₁-C₆ alkyl, C₁-C₆ alkyl halide, C₁-C₆ alkoxy, and C₁-C₆ alkylamine or R³ and R⁴ or R⁴ and R⁵ or R⁵ and R⁶ or R⁶ and R⁷ combine together with the atoms to which they are attached form a C₅-C₈ aryl or heteroaryl, or C₅-C₈ cycloalkenyl or heterocycloalkenyl,
wherein R³ to R⁷ are independently unsubstituted or substituted with hydroxyl, halogen, C₁-C₃ alkyl, C₁-C₃ alkenyl, or C₁-C₃ alkyl halide.

In certain embodiments of Formula I, the compound can be represented by a structure having the Formula I-B-1:

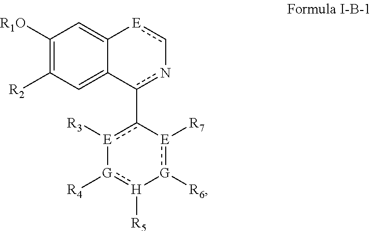

wherein
R¹ is selected from hydrogen and C₁-C₆ alkyl;
R² is selected from hydrogen, hydroxyl, C₁-C₆ alkyl, and C₁-C₆ alkoxy; and
R³ to R⁷ are independently selected from hydrogen, hydroxyl, C₁-C₆ alkyl, C₁-C₆ alkyl halide, C₁-C₆ alkoxy, and C₁-C₆ alkylamine or R³ and R⁴ or R⁴ and R⁵ or R⁵ and R⁶ or R⁶ and R⁷ combine together with the atoms to which they are attached form a C₅-C₈ aryl or heteroaryl, or C₅-C₈ cycloalkenyl or heterocycloalkenyl,
wherein R³ to R⁷ are independently unsubstituted or substituted with hydroxyl, halogen, C₁-C₃ alkyl, C₁-C₃ alkenyl, or C₁-C₃ alkyl halide.

In certain embodiments of Formula I, the compound can be represented by a structure having the Formula I′-B-1:

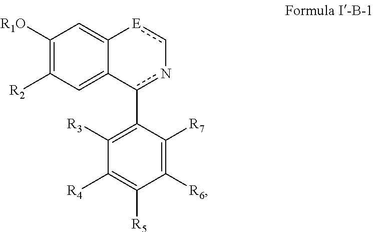

wherein
R¹ is selected from hydrogen and C₁-C₆ alkyl;
R² is selected from hydrogen, hydroxyl, C₁-C₆ alkyl, and C₁-C₆ alkoxy; and
R³ to R⁷ are independently selected from hydrogen, hydroxyl, C₁-C₆ alkyl, C₁-C₆ alkyl halide, C₁-C₆ alkoxy, and C₁-C₆ alkylamine or R³ and R⁴ or R⁴ and R⁵ or R⁵ and R⁶ or R⁶ and R⁷ combine together with the atoms to which they are attached form a C₅-C₈ aryl or heteroaryl, or C₅-C₈ cycloalkenyl or heterocycloalkenyl,
wherein R³ to R⁷ are independently unsubstituted or substituted with hydroxyl, halogen, C₁-C₃ alkyl, C₁-C₃ alkenyl, or C₁-C₃ alkyl halide.

In certain embodiments of Formula I, the compound can be represented by a structure having the Formula I-C-1:

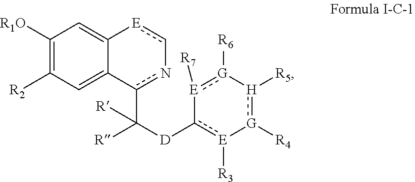

wherein
D is selected from CR′R″, NR′, and O,
R′ and R″ are independently selected from hydrogen, hydroxyl, halogen, amine, alkylamine, C₁-C₆ alkyl, C₁-C₆ alkyl halide, C₁-C₆ alkoxy, or R′ and R″ combine together with the atom to which they are attached form a carbonyl;
R¹ is selected from hydrogen and C₁-C₆ alkyl;
R² is selected from hydrogen, hydroxyl, C₁-C₆ alkyl, and C₁-C₆ alkoxy; and
R³ to R⁷ are independently selected from hydrogen, hydroxyl, C₁-C₆ alkyl, C₁-C₆ alkyl halide, C₁-C₆ alkoxy, and C₁-C₆ alkylamine or R³ and R⁴ or R⁴ and R⁵ or R⁵ and R⁶ or R⁶ and R⁷ combine together with the atoms to which they are attached form a C₅-C₈ aryl or heteroaryl, or C₅-C₈ cycloalkenyl or heterocycloalkenyl,
wherein R³ to R⁷ are independently unsubstituted or substituted with hydroxyl, halogen, C₁-C₃ alkyl, C₁-C₃ alkenyl, or C₁-C₃ alkyl halide.

In certain embodiments of Formula I, the compound can be represented by a structure having the Formula I′-C-1:

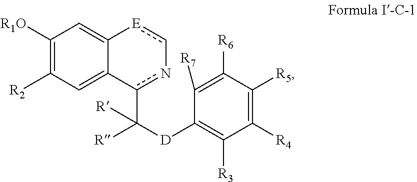

wherein
D is selected from CR′R″, NR′, and O,
R′ and R″ are independently selected from hydrogen, hydroxyl, halogen, amine, alkylamine, C₁-C₆ alkyl, C₁-C₆ alkyl halide, C₁-C₆ alkoxy, or R′ and R″ combine together with the atom to which they are attached form a carbonyl;
R¹ is selected from hydrogen and C₁-C₆ alkyl;
R² is selected from hydrogen, hydroxyl, C₁-C₆ alkyl, and C₁-C₆ alkoxy; and
R³ to R⁷ are independently selected from hydrogen, hydroxyl, C₁-C₆ alkyl, C₁-C₆ alkyl halide, C₁-C₆ alkoxy, and C₁-C₆ alkylamine or R³ and R⁴ or R⁴ and R⁵ or R⁵ and R⁶ or R⁶ and R⁷ combine together with the atoms to which they are attached form a C₅-C₈ aryl or heteroaryl, or C₅-C₈ cycloalkenyl or heterocycloalkenyl,
wherein R³ to R⁷ are independently unsubstituted or substituted with hydroxyl, halogen, C₁-C₃ alkyl, C₁-C₃ alkenyl, or C₁-C₃ alkyl halide.

In certain embodiments, the compound can be represented by a structure having the Formula II:

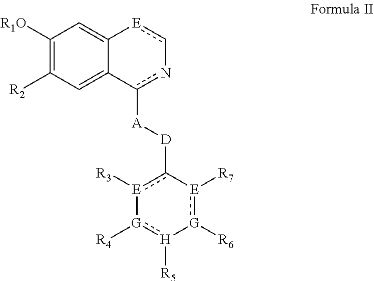

wherein
A and D are independently present or absent and are independently selected from CR′R″, NR′, and O, wherein R′ and R″ are independently for each occurrence selected from hydrogen, hydroxyl, halogen, amine, alkylamine, C₁-C₆ alkyl, C₁-C₆ alkyl halide, C₁-C₆ alkoxy, or R′ and R″ combine together with the atom to which they are attached form a carbonyl group;
E, G, and H can be independently selected from C, N′, O, and S;
R¹ and R² are independently selected from hydrogen, C₁-C₆ alkyl, C₁-C₆ alkyl halide, C₁-C₆ alkoxy, and C₁-C₆ alkylamine;
R³ to R⁷ are independently selected from hydrogen, hydroxyl, C₁-C₆ alkyl, C₁-C₆ alkyl halide, C₁-C₆ alkoxy, and C₁-C₆ alkylamine or R³ and R⁴ or R⁴ and R⁵ or R⁵ and R⁶ or R⁶ and R⁷ combine together with the atoms to which they are attached form a C₅-C₈ aryl or heteroaryl, or C₅-C₈ cycloalkenyl or heterocycloalkenyl; wherein R³ to R⁷ are independently unsubstituted or substituted with hydroxyl, halogen, C₁-C₃ alkyl, C₁-C₃ alkenyl, or C₁-C₃ alkyl halide; and
----- represents a bond and is independently for each occurrence absent or present.

In some embodiments of the formulas described herein (including Formulas I, I′, I-A to I-C, I′-A to I′-C, I-A-1 to I-C-1, I′-A-1 to I′-C-1, and II), A can be selected from CR′R″ and O, wherein R′ and R″ are as defined herein. In some embodiments of the formulas described herein, D is selected from CR′R″ and O, wherein R′ and R″ are as defined herein. In some embodiments of the formulas described herein, A and D can both be CR′R″. In some embodiments of the formulas described herein, A can be CR′R″ and D is O. In some embodiments of the formulas described herein, A is present and D is absent.

In some embodiments of the formulas described herein, R′ and R″ can be independently for each occurrence selected from hydrogen, hydroxyl, C₁-C₆ alkyl, or R′ and R″ combine together with the atom to which they are attached can form a carbonyl. For example, R′ and R″ can be hydrogen. In some examples, R′ can be hydrogen and at least one occurrence of R″ can be hydroxyl. In other examples, at least one occurrence of R′ and R″ combine together with the atom to which they are attached form a carbonyl.

In some embodiments of the formulas described herein, R¹ can be selected from a C₁-C₆ alkyl. For example, R¹ can be selected from a C₁-C₂ alkyl such as methyl or ethyl. In some examples, R¹ can be methyl.

In some embodiments of the formulas described herein, R² can be independently selected from a C₁-C₆ alkoxy. For example, R² is selected from a C₁-C₂ alkoxy such as methoxy or ethoxy. In some examples, R² can be methoxy. In other examples, R² can be hydrogen.

In some embodiments of the formulas described herein, R³, R⁴, R⁵, R⁶, and R⁷ can be independently selected from hydrogen, hydroxyl, C₁-C₆ alkyl, C₁-C₆ alkyl halide, C₁-C₆ alkoxy, or R³ and R⁴ or R⁴ and R⁵ combine together with the atoms to which they are attached form a C₅-C₈ aryl or heteroaryl, or C₅-C₈ cycloalkenyl or heterocycloalkenyl. For example, R³ to R⁷ can be independently unsubstituted or substituted with hydroxyl, halogen, C₁-C₃ alkyl, C₁-C₃ alkenyl, or C₁-C₃ alkyl halide. In some examples, R³ and R⁷ are hydrogen. In some examples, R⁴, R⁵, and R⁶ are independently selected from hydrogen, hydroxyl, C₁-C₆ alkyl, C₁-C₆ alkyl halide, or C₁-C₆ alkoxy. In further examples, R³ and R⁴ or R⁴ and R⁵ combine together with the atoms to which they are attached form a C₆ aryl, a C₆ heteroaryl, or a C₅ heterocycloalkenyl.

In certain embodiments of the formulas described herein, the compound can be represented by a structure below:

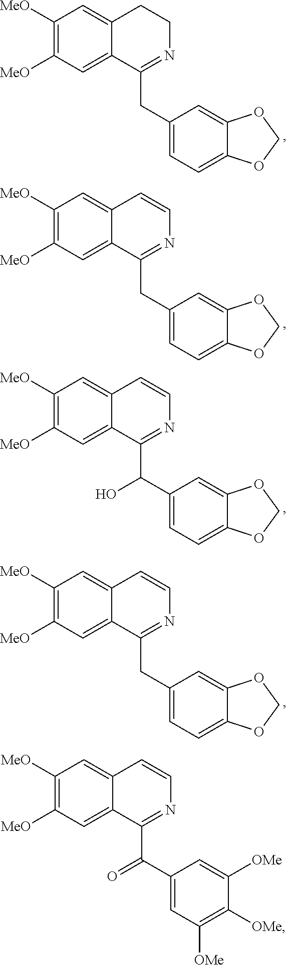

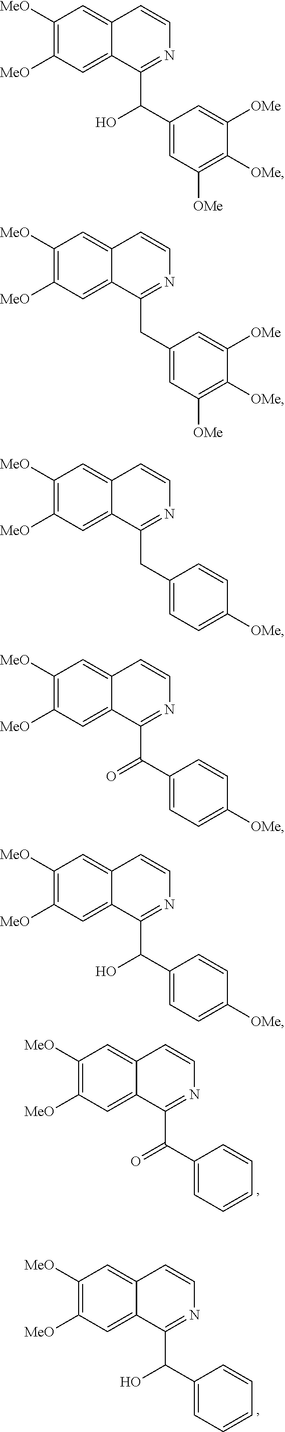

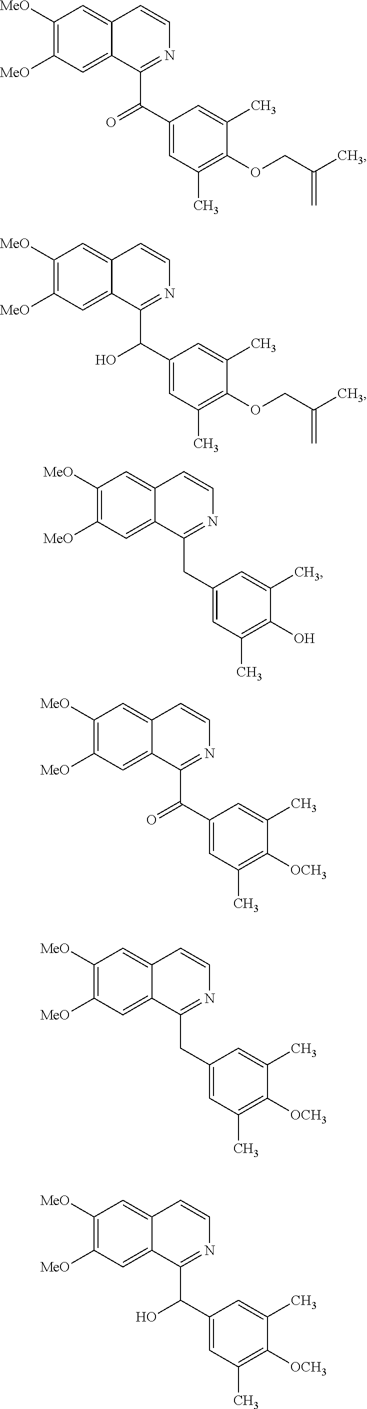

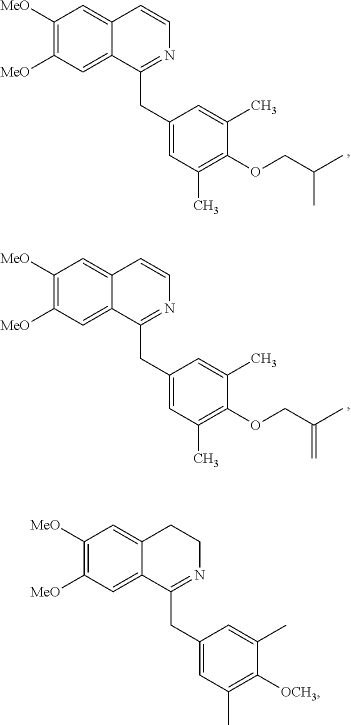 [SMILES: COc1cc2ccnc(Cc3cc(C)c(OCC(C)C)c(C)c3)c2cc1OC ; C=C(C)COc1c(C)cc(Cc2nccc3cc(OC)c(OC)cc23)cc1C ; COc1cc2c(cc1OC)C(Cc1cc(C)c(OC)c(C)c1)=NCC2]

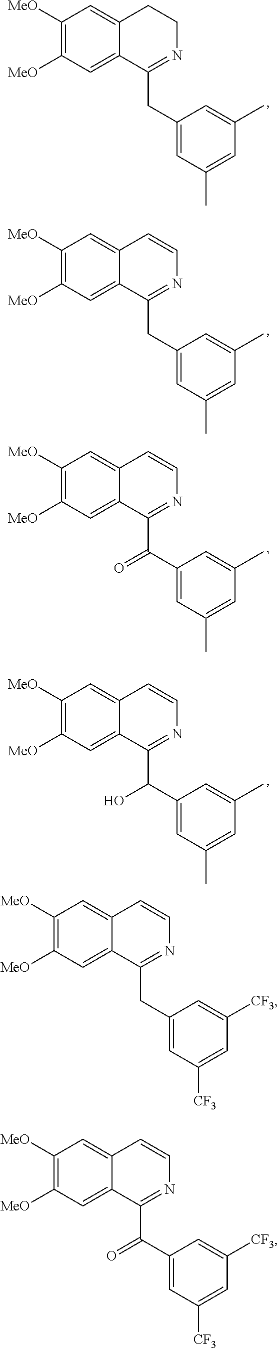

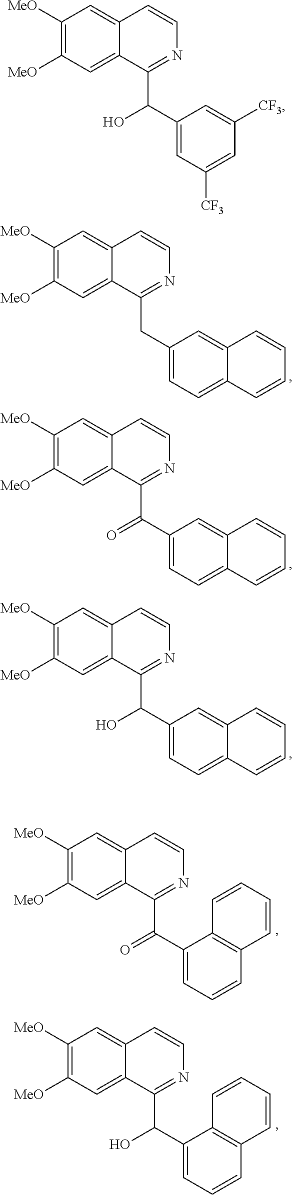 [SMILES: COc1cc2ccnc(C(O)c3cc(C(F)(F)F)cc(C(F)(F)F)c3)c2cc1OC ; COc1cc2ccnc(Cc3ccc4ccccc4c3)c2cc1OC ; COc1cc2ccnc(C(=O)c3ccc4ccccc4c3)c2cc1OC ; COc1cc2ccnc(C(O)c3ccc4ccccc4c3)c2cc1OC ; COc1cc2ccnc(C(=O)c3cccc4ccccc34)c2cc1OC ; COc1cc2ccnc(C(O)c3cccc4ccccc34)c2cc1OC]

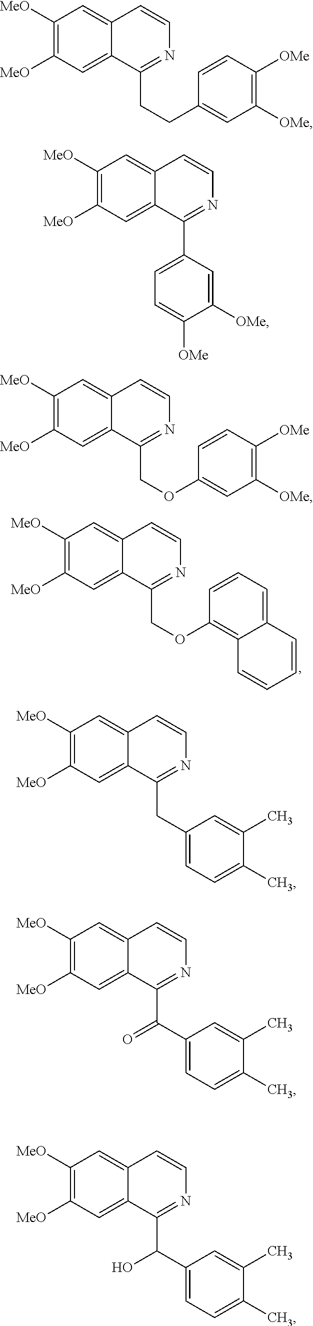

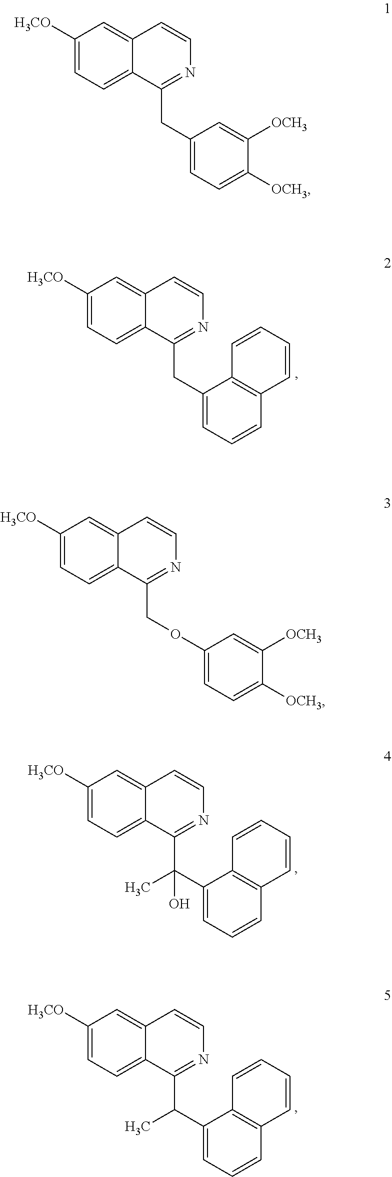 [SMILES: COc1ccc2c(Cc3ccc(OC)c(OC)c3)nccc2c1 ; COc1ccc2c(Cc3cccc4ccccc34)nccc2c1 ; COc1ccc2c(COc3ccc(OC)c(OC)c3)nccc2c1 ; COc1ccc2c(C(C)(O)c3cccc4ccccc34)nccc2c1 ; COc1ccc2c(C(C)c3cccc4ccccc34)nccc2c1]

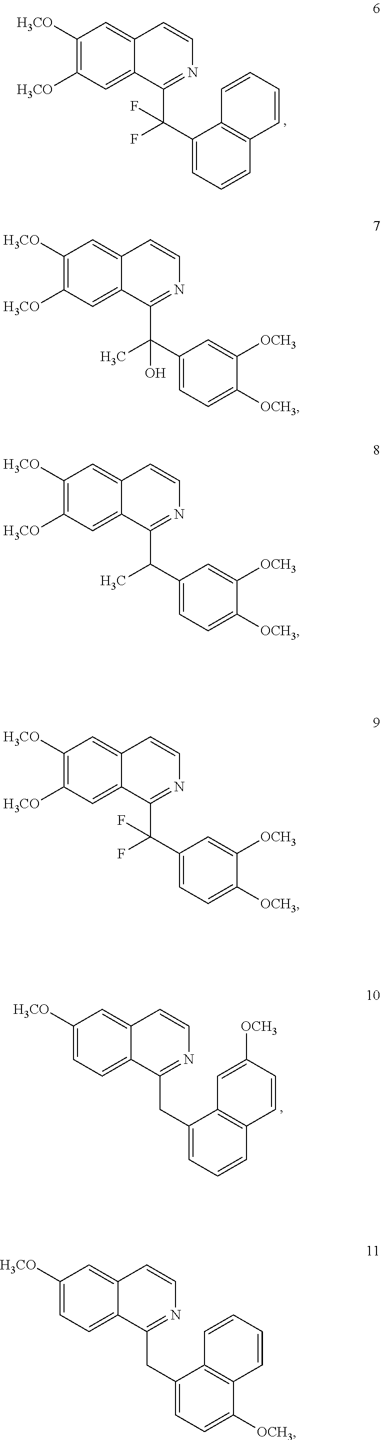 [SMILES: COc1cc2ccnc(C(F)(F)c3cccc4ccccc34)c2cc1OC ; COc1ccc(C(C)(O)c2nccc3cc(OC)c(OC)cc23)cc1OC ; COc1ccc(C(C)c2nccc3cc(OC)c(OC)cc23)cc1OC ; COc1ccc(C(F)(F)c2nccc3cc(OC)c(OC)cc23)cc1OC ; COc1ccc2c(Cc3cccc4ccc(OC)cc34)nccc2c1 ; COc1ccc2c(Cc3ccc(OC)c4ccccc34)nccc2c1]

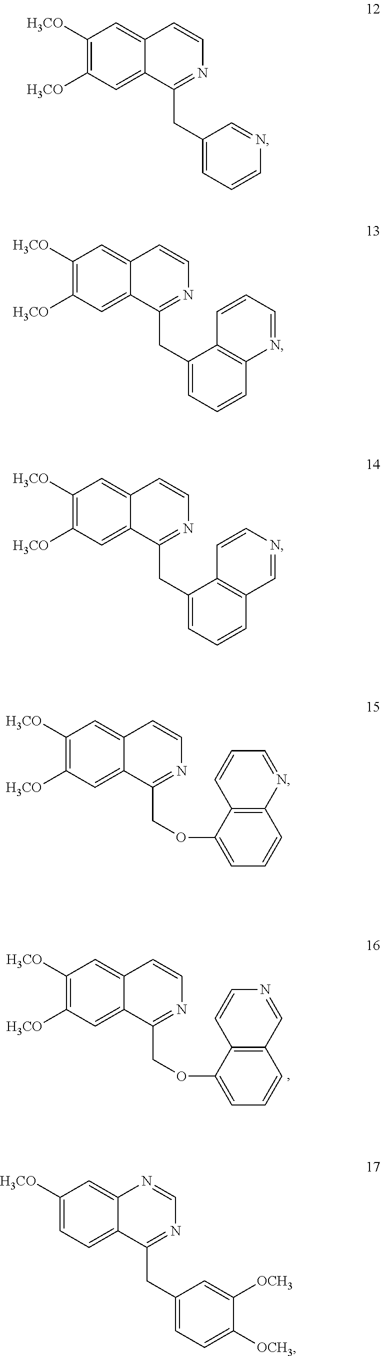 [SMILES: COc1cc2ccnc(Cc3cccnc3)c2cc1OC ; COc1cc2ccnc(Cc3cccc4ncccc34)c2cc1OC ; COc1cc2ccnc(Cc3cccc4cnccc34)c2cc1OC ; COc1cc2ccnc(COc3cccc4ncccc34)c2cc1OC ; COc1cc2ccnc(COc3cccc4cnccc34)c2cc1OC ; COc1ccc2c(Cc3ccc(OC)c(OC)c3)ncnc2c1]

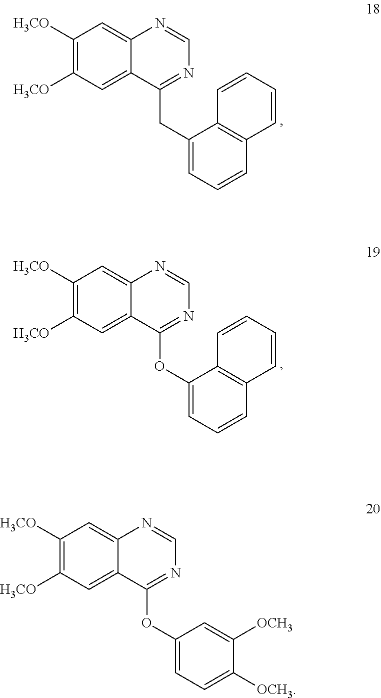 [SMILES: COc1cc2ncnc(Cc3cccc4ccccc34)c2cc1OC ; COc1cc2ncnc(Oc3cccc4ccccc34)c2cc1OC ; COc1ccc(Oc2ncnc3cc(OC)c(OC)cc23)cc1OC]

Pharmaceutical Compositions

The disclosed compounds can be used therapeutically in combination with a pharmaceutically acceptable carrier. The carrier would naturally be selected to minimize any degradation of the active ingredient and to minimize any adverse side effects in the subject, as would be well known to one of skill in the art.

The disclosed compounds may be in solution, suspension, incorporated into microparticles, liposomes, or cells, or formed into tablets, gels, or suppositories. Suitable carriers and their formulations are described in Remington: The Science and Practice of Pharmacy (22^{nd} ed.) eds. Loyd V. Allen, Jr., et al., Pharmaceutical Press, 2012. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered. Pharmaceutical carriers are known to those skilled in the art. These most typically would be standard carriers for administration of vaccines to humans, including solutions such as sterile water, saline, and buffered solutions at physiological pH. Pharmaceutical compositions may include carriers, thickeners, diluents, buffers, preservatives, surface active agents and the like in addition to the vaccine. Pharmaceutical compositions may also include one or more active ingredients such as antimicrobial agents, anti-inflammatory agents, anesthetics, and the like.

The disclosed compounds are preferably formulated for delivery via intranasal, intramuscular, subcutaneous, parenteral, transdermal or sublingual administration.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like. Parenteral administration of the disclosed compounds, if used, is generally characterized by injection. Injectables can be prepared in conventional forms, either as liquid solutions or suspensions, solid forms suitable for solution of suspension in liquid prior to injection, or as emulsions.

For an oral administration form, the disclosed compounds can be mixed with suitable additives, such as excipients, stabilizers or inert diluents, and brought by means of the customary methods into the suitable administration forms, such as tablets, coated tablets, hard capsules, aqueous, alcoholic, or oily solutions. Examples of suitable inert carriers are gum arabic, magnesia, magnesium carbonate, potassium phosphate, lactose, glucose, or starch, in particular, cornstarch. In this case, the preparation can be carried out both as dry and as moist granules. Suitable oily excipients or solvents are vegetable or animal oils, such as sunflower oil or cod liver oil. Suitable solvents for aqueous or alcoholic solutions are water, ethanol, sugar solutions, or mixtures thereof. Polyethylene glycols and polypropylene glycols are also useful as further auxiliaries for other administration forms. As immediate release tablets, these compositions may contain microcrystalline cellulose, dicalcium phosphate, starch, magnesium stearate and lactose and/or other excipients, binders, extenders, disintegrants, diluents and lubricants known in the art.

When administered by nasal aerosol or inhalation, the disclosed compounds may be prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art. Suitable pharmaceutical formulations for administration in the form of aerosols or sprays are, for example, solutions, suspensions or emulsions of the compounds of the disclosure or their physiologically tolerable salts in a pharmaceutically acceptable solvent, such as ethanol or water, or a mixture of such solvents. If required, the formulation may additionally contain other pharmaceutical auxiliaries such as surfactants, emulsifiers and stabilizers as well as a propellant.

For subcutaneous or intravenous administration, the disclosed compounds, if desired with the substances customary therefore such as solubilizers, emulsifiers or further auxiliaries are brought into solution, suspension, or emulsion. The disclosed compounds may also be lyophilized and the lyophilizates obtained used, for example, for the production of injection or infusion preparations. Suitable solvents are, for example, water, physiological saline solution or alcohols, e.g. ethanol, propanol, glycerol, sugar solutions such as glucose or mannitol solutions, or mixtures of the various solvents mentioned. The injectable solutions or suspensions may be formulated according to known art, using suitable non-toxic, parenterally-acceptable diluents or solvents, such as mannitol, 1,3-butanediol, water, Ringer's solution or isotonic sodium chloride solution, or suitable dispersing or wetting and suspending agents, such as sterile, bland, fixed oils, including synthetic mono- or diglycerides, and fatty acids, including oleic acid.

When rectally administered in the form of suppositories, the formulations may be prepared by mixing the compounds with a suitable non-irritating excipient, such as cocoa butter, synthetic glyceride esters or polyethylene glycols, which are solid at ordinary temperatures, but liquefy and/or dissolve in the rectal cavity to release the drug.

In certain embodiments, it is contemplated that compositions comprising the disclosed compounds can be extended release formulations. Typical extended release formations utilize an enteric coating. Typically, a barrier is applied to oral medication that controls the location in the digestive system where it is absorbed. Enteric coatings prevent release of medication before it reaches the small intestine. Enteric coatings may contain polymers of polysaccharides, such as maltodextrin, xanthan, scleroglucan dextran, starch, alginates, pullulan, hyaloronic acid, chitin, chitosan and the like; other natural polymers, such as proteins (albumin, gelatin etc.), poly-L-lysine; sodium poly(acrylic acid); poly(hydroxyalkylmethacrylates) (for example poly(hydroxyethylmethacrylate)); carboxypolymethylene (for example Carbopol™); carbomer; polyvinylpyrrolidone; gums, such as guar gum, gum arabic, gum karaya, gum ghatti, locust bean gum, tamarind gum, gellan gum, gum tragacanth, agar, pectin, gluten and the like; poly(vinyl alcohol); ethylene vinyl alcohol; polyethylene glycol (PEG); and cellulose ethers, such as hydroxymethylcellulose (HMC), hydroxyethylcellulose (HEC), hydroxypropylcellulose (HPC), methylcellulose (MC), ethylcellulose (EC), carboxyethylcellulose (CEC), ethylhydroxyethylcellulose (EHEC), carboxymethylhydroxyethylcellulose (CMHEC), hydroxypropylmethyl-cellulose (HPMC), hydroxypropylethylcellulose (HPEC) and sodium carboxymethylcellulose (Na-CMC); as well as copolymers and/or (simple) mixtures of any of the above polymers. Certain of the above-mentioned polymers may further be crosslinked by way of standard techniques.

The choice of polymer will be determined by the nature of the active ingredient/drug that is employed in the composition of the disclosure as well as the desired rate of release. In particular, it will be appreciated by the skilled person, for example in the case of HPMC, that a higher molecular weight will, in general, provide a slower rate of release of drug from the composition. Furthermore, in the case of HPMC, different degrees of substitution of methoxyl groups and hydroxypropoxyl groups will give rise to changes in the rate of release of drug from the composition. In this respect, and as stated above, it may be desirable to provide compositions of the disclosure in the form of coatings in which the polymer carrier is provided by way of a blend of two or more polymers of, for example, different molecular weights in order to produce a particular required or desired release profile.

Microspheres of polylactide, polyglycolide, and their copolymers poly(lactide-co-glycolide) may be used to form sustained-release delivery systems. The disclosed compounds can be entrapped in the poly(lactide-co-glycolide) microsphere depot by a number of methods, including formation of a water-in-oil emulsion with water-borne compound and organic solvent-borne polymer (emulsion method), formation of a solid-in-oil suspension with solid compound dispersed in a solvent-based polymer solution (suspension method), or by dissolving the compound in a solvent-based polymer solution (dissolution method). One can attach poly(ethylene glycol) to compounds (PEGylation) to increase the in vivo half-life of circulating therapeutic proteins and decrease the chance of an immune response.

Liposomal suspensions (including liposomes targeted to viral antigens) may also be prepared by conventional methods to produce pharmaceutically acceptable carriers. This may be appropriate for the delivery of free nucleosides, acyl nucleosides or phosphate ester prodrug forms of the nucleoside compounds according to the present disclosure.

The exact amount of the compounds or compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein. For example, effective dosages and schedules for administering the compositions may be determined empirically, and making such determinations is within the skill in the art. The dosage ranges for the administration of the compositions are those large enough to produce the desired effect in which the symptoms disorder are affected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counter indications. Dosage can vary, and can be administered in one or more dose administrations daily, for one or several days. Guidance can be found in the literature for appropriate dosages for given classes of pharmaceutical products. A typical dosage of the disclosed vaccine used alone might range from about 1 μg/kg to up to 100 mg/kg of body weight or more per vaccination, such as 10 μg/kg to 50 mg/kg, or 50 μg/kg to 10 mg/kg, depending on the factors mentioned above.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Some of the disclosed compounds may potentially be administered as a pharmaceutically acceptable acid- or base- addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

The pharmaceutical preparations of the disclosure are preferably in a unit dosage form, and may be suitably packaged, for example in a box, blister, vial, bottle, sachet, ampoule or in any other suitable single-dose or multi-dose holder or container (which may be properly labeled); optionally with one or more leaflets containing product information and/or instructions for use. Generally, such unit dosages will contain between 1 and 1000 mg, and usually between 5 and 500 mg, of the at least one compound of the disclosure, e.g., about 10, 25, 50, 100, 200, 300 or 400 mg per unit dosage.

The disclosed compounds can also be used to supplement existing treatments. Therefore, the disclosed compositions can further include (or be administered in combination with) a second compound that can ameliorate, diminishing, reversing, treating or preventing cancer in a subject. For example, the disclosed compositions can further include (or be administered in combination with) one or more chemotherapeutic agents. In a specific embodiment, the disclosed compounds can be administered with (in combination in the same composition, in combination but in separate compositions, or sequentially) approved drugs for treating cancer.

The pharmaceutical compositions and formulations disclosed herein can be administered for prophylactic and/or therapeutic treatments. In therapeutic applications, compositions are administered to a subject already having a tumor.

The amount of pharmaceutical composition adequate to accomplish this is defined as a “therapeutically effective dose.” The dosage schedule and amounts effective for this use, i.e., the “dosing regimen,” will depend upon a variety of factors, including the stage of the condition, the severity of the condition, the general state of the patient's health, the patient's physical status, age and the like. In calculating the dosage regimen for a patient, the mode of administration also is taken into consideration.

The dosage regimen also takes into consideration pharmacokinetics parameters well known in the art, i.e., the active agents' rate of absorption, bioavailability, metabolism, clearance, and the like (see, e.g., Hidalgo-Aragones (1996) J. Steroid Biochem. Mol. Biol. 58:611-617; Groning (1996) Pharmazie 51:337-341; Fotherby (1996) Contraception 54:59-69; Johnson (1995) J. Pharm. Sci. 84:1144-1146; Rohatagi (1995) Pharmazie 50:610-613; Brophy (1983) Eur. J. Clin. Pharmacol. 24:103-108; the latest Remington's, supra). The state of the art allows the clinician to determine the dosage regimen for each individual patient, active agent and disease or condition treated. Guidelines provided for similar compositions used as pharmaceuticals can be used as guidance to determine the dosage regiment, i.e., dose schedule and dosage levels, administered practicing the methods of the invention are correct and appropriate.

Methods of Use

The present disclosure provides methods for treating or ameliorating at least one symptom or indication, or inhibiting the growth of a locally advanced, surgically undesirable, or metastatic malignant melanoma in a subject cancer in a subject. In certain embodiments, the present disclosure provides methods for inhibiting mitochondrial oxygen consumption in a cancerous tissue. In specific embodiments, the present disclosure provides methods for treating or ameliorating at least one symptom or indication, or inhibiting the growth of hypoxic tumors. Hypoxic tumors exists because the supply of oxygen is insufficient to meet the metabolic demand of the tumor.

The methods can comprise administering to a subject in need thereof an effective amount of a compound or composition disclosed herein. The compound or composition can be in an effective amount to reduce oxygen consumption in the tumor cells. In some embodiments, the compounds and compositions can be in an effective amount to inhibit mitochondrial functions in the tumor cell. In some embodiments, the compounds or compositions disclosed herein can be in an effective amount to inhibit complex 1 of the mitochondrial respiratory chain. In some embodiments, the compounds or compositions can be in an effective amount to inhibit phosphodiesterase 10A (PDE10A). In some embodiments, the compounds or compositions are not PDE10A inhibitors. In some examples, the compounds and compositions for treating or reducing tumor hypoxia includes papaverine.

In certain embodiments, the compound or compositions disclosed herein can be administered to a subject in need thereof, each dose comprising 0.1-10 mg/kg (e.g., 0.3 mg/kg, 1 mg/kg, 3 mg/kg, or 10 mg/kg) of the subject's body weight. In certain other embodiments, each dose comprises 20-600 mg of the compound, e.g., 50 mg, 100 mg, 150 mg, 200 mg, 250 mg, 300 mg, 400 mg, or 500 mg of the compound.

The methods of treating hypoxic tumors in a subject can include administering the compound or compositions disclosed herein in combination with radiation therapy. For example, the method can further comprise irradiating the cancerous tissue with an ionizing radiation for an effective period.

As used herein, the term “radiation therapy”, also referred to as “XRT” means using ionizing radiation to kill cancer cells, generally as part of anti-cancer therapy. X-rays, gamma rays or charged particles (e.g., protons or electrons) are used to generate ionizing radiation. Radiation therapy can be delivered by a machine placed outside the subject's body (external-beam radiation therapy), or by a source placed inside a subject's body (internal radiation therapy or brachytherapy), or through systemic radioisotopes delivered intravenously or orally (systemic radioisotope therapy). Radiation therapy can be planned and administered in conjunction with imaging-based techniques such a computed tomography (CT), magnetic resonance imaging (MRI) to accurately determine the dose and location of radiation to be administered. In various embodiments, radiation therapy can be selected from the group consisting of total all-body radiation therapy, conventional external beam radiation therapy, stereotactic radiosurgery, stereotactic body radiation therapy, 3-D conformal radiation therapy, intensity-modulated radiation therapy, image-guided radiation therapy, tomotherapy, brachytherapy, and systemic radiation therapy. Depending upon the intent, in certain embodiments, radiation therapy is curative, adjuvinating or palliative.

In specific embodiments, the term “radiation therapy” refers to hypofractionated radiation therapy. Hypofractionated radiation therapy refers to radiation therapy in which a radiation dose is comprised in 1 or more fractions. In various embodiments, each fraction can comprise 2-20 Gy. For example, a radiation dose of 50 Gy may be split up into 10 fractions, each comprising 5 Gy. In certain embodiments, the 1 or more fractions can be administered on consecutive or sequential days. In certain other embodiments, the 2 or more fractions are administered once in 2 days, once in 3 days, once in 4 days, once in 5 days, once in 6 days, once in 7 days, or in a combination thereof.

The term “Gray (Gy)” as used herein refers to a derived metric (SI) measurement unit of absorbed radiation dose of ionizing radiation, e.g. x-rays, and is defined as the absorption of one joule of ionizing radiation by one kilogram (1J/kg) of matter, e.g. human tissue.

In specific embodiments, the methods for treating hypoxic tumors in a subject comprises administering radiation therapy in a total dose of 25 Gray or greater (for e.g., 30 Gray or greater, 35 Gray or greater, 40 Gray or greater, 45 Gray or greater, 50 Gray or greater, 55 Gray or greater, 60 Gray or greater, 65 Gray or greater, 70 Gray or greater, 75 Gray or greater, or from 25 Gray to 75 Gray). The cancerous tissue can be irradiated with at least 1 fraction (for e.g, 2 fractions or greater, 4 fractions or greater, 5 fractions or greater, 6 fractions or greater, 7 fractions or greater, 8 fractions or greater, 9 fractions or greater, 10 fractions or greater, 15 fractions or greater, 20 fractions or greater, 25 fractions or greater, 30 fractions or greater, from 1 fraction to 30 fractions, or from 2 fractions to 20 fractions), for a total from 25 to 75 Gray. In certain embodiments, the radiation treatment can be hypofractionated. For example, the cancerous tissue can be irradiated with from 1 to 6 fractions of radiation per day, the total fraction of radiation being from about 40 to about 75 Gray.

In certain embodiments, the method comprises administering one or more doses in a treatment cycle. For example, the method can comprise administering to a subject in need thereof at least one treatment cycle, wherein the at least one treatment cycle comprises 1-10 doses of a compound or composition disclosed herein and optionally one or more doses of radiation therapy. In certain embodiments, 2-12 treatment cycles are administered to a subject in need thereof.

In specific embodiments, the present disclosure provides methods for increased anti-tumor efficacy or increased tumor inhibition. The methods can comprise administering to a subject with a solid tumor a therapeutically effective amount of a compound or composition disclosed herein prior to administering a radiation dose, wherein the compound or composition can be administered on the same day as the ionizing radiation is administered. In some embodiments, the compound or composition can be administered 6 hours or less, 5 hours or less, 4 hours or less, 3 hours or less, 2 hours or less, 1 hour or less, or 0.5 hours or less, prior to the radiation therapy. In certain embodiments, the methods provide for increased tumor inhibition, e.g., by about 20%, more than 20%, more than 30%, more than 40% more than 50%, more than 60%, more than 70% or more than 80% as compared to a subject administered with a radiation dose absent a compound or compositions disclosed herein. In certain embodiments, the radiation therapy comprises hypofractionated radiation therapy.

In certain embodiments, the cancer or tumor is a solid tumor or malignancy. The methods described herein can cause a therapeutic injury resulting in the reduction of at least one of surface area, the depth, and the amount of the tissue affected by the cancerous condition. In certain embodiments, the compounds and compositions can be used in the treatment of cancer of the bile duct, bone, bladder, head and neck, kidney, liver, gastrointestinal tissue, oesophagus, ovary, endometrium, pancreas, skin, testes, thyroid, uterus, cervix and vulva, and of leukaemias (including ALL and CML), multiple myeloma and lymphomas. In specific embodiments, the compounds and compositions can be used in the treatment of lung cancer, anal cancer, colorectal cancer, prostate cancer, melanoma, renal cancer, skin cancer, testicular cancer, ovarian cancer, breast cancer, endometrial cancer, kidney cancer, gastric cancer, sarcomas, bladder cancer, brain cancer, cervical cancer, gastrointestinal cancer, genitourinary cancer, esophageal cancer, pancreatic cancer, colon cancer, liver cancer, uterine cancer, bone cancer, stomach cancer, salivary gland cancer, head and neck cancers, tumors of the central nervous system and their metastases, and also for the treatment of glioblastomas and myeloma. In some specific embodiments, the cancer is lung cancer.

In some embodiments, compounds and compositions disclosed herein could be used in the clinic either as a single agent by itself, in combination with radiation, or in combination with both radiation and an additional chemotherapy agent. Such chemotherapy agent can include one or more of the following categories of anti-tumour agents:

(i) antiproliferative/antineoplastic drugs and combinations thereof, as used in medical oncology, such as alkylating agents (for example cis-platin, carboplatin, cyclophosphamide, nitrogen mustard, melphalan, chlorambucil, busulfan and nitrosoureas); antimetabolites (for example antifolates such as fluoropyrimidines like 5-fluorouracil and gemcitabine, tegafur, raltitrexed, methotrexate, cytosine arabinoside and hydroxyurea); antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere); and topoisomerase inhibitors (for example epipodophyllotoxins like etoposide and teniposide, amsacrine, topotecan and camptothecin); and proteosome inhibitors (for example bortezomib [Velcade®]); and the agent anegrilide [Agrylin®]; and the agent alpha-interferon;

(ii) cytostatic agents such as anti-estrogens (for example tamoxifen, toremifene, raloxifene, droloxifene and iodoxyfene), oestrogen receptor down regulators (for example fulvestrant), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5α-reductase such as finasteride;

(iii) agents that inhibit cancer cell invasion (for example metalloproteinase inhibitors like marimastat and inhibitors of urokinase plasminogen activator receptor function); (iv) inhibitors of growth factor function, for example such inhibitors include growth factor antibodies, growth factor receptor antibodies (for example the anti-erbb2 antibody trastuzumab [Herceptin™] and the anti-erbb1 antibody cetuximab), farnesyl transferase inhibitors, tyrosine kinase inhibitors and serine/threonine kinase inhibitors, for example inhibitors of the epidermal growth factor family (for example EGFR family tyrosine kinase inhibitors such as: N-(3-chloro-4-fluorophenyl)-7-methoxy-6-(3-morpholinopropoxy)quinazolin-4-a mine (gefitinib), N-(3-ethynylphenyl)-6,7-bis(2-methoxyethoxy)quinazolin-4-amine (erlotinib), and 6-acrylamido-N-(3-chloro-4-fluorophenyl)-7-(3-morpholinopropoxy)quinazolin-4-amine (CI 1033), for example inhibitors of the platelet-derived growth factor family and for example inhibitors of the hepatocyte growth factor family, for example inhibitors or phosphotidylinositol 3-kinase (PI3K) and for example inhibitors of mitogen activated protein kinase kinase (MEK1/2) and for example inhibitors of protein kinase B (PKB/Akt), for example inhibitors of Src tyrosine kinase family and/or Abelson (AbI) tyrosine kinase family such as dasatinib (BMS-354825) and imatinib mesylate (Gleevec™); and any agents that modify STAT signalling;

(v) antiangiogenic agents such as those which inhibit the effects of vascular endothelial growth factor, (for example the anti-vascular endothelial cell growth factor antibody bevacizumab [Avastin™]) and compounds that work by other mechanisms (for example linomide, inhibitors of integrin ocvP3 function and angiostatin);

(vi) vascular damaging agents such as Combretastatin A4;

(vii) antisense therapies, for example those which are directed to the targets listed above, such as an anti-ras antisense;

(viii) gene therapy approaches, including for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy; and

(ix) immunotherapy approaches, including for example ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies, and approaches using the immunomodulatory drugs thalidomide and lenalidomide [Revlimid®].

Combination treatment with an additional chemotherapy agent can be achieved by way of the simultaneous, sequential or separate dosing of the individual components of the treatment. Such combination products employ the compounds or compositions of this disclosure, or pharmaceutically acceptable salts thereof, within the dosage range described hereinbefore and the other pharmaceutically-active agent within its approved dosage range.

The methods described herein are provided for treating or ameliorating at least one symptom or indication, or inhibiting the growth of cancer in a subject. In certain embodiments, the methods described herein are provided for treating or ameliorating at least one symptom or indication, or inhibiting the growth of hypoxic cancer in a subject. In certain embodiments, methods are provided for increasing the overall or progression-free survival of a patient with cancer. In some embodiments, the compounds and compositions are effective radiosensitizer of subcutaneous and orthotopic tumors.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the scope of the disclosure. Unless indicated otherwise, parts are parts by weight, temperature is in ° C. or is at ambient temperature, and pressure is at or near atmospheric.

### Example 1: Repurposing Papaverine to Metabolically Radiosensitize Hypoxic Tumors

Abstract: Hypoxia is a common microenvironmental feature of solid tumors (Brown, J. M. et al. Cancer Res 58, 1408-1416 (1998)) that exists because the supply of oxygen is insufficient to meet the metabolic demand of the tumor (Epstein, T. et al. Cancer Metab 2, 7 (2014) and Semenza, G. L. et al. The Journal of clinical investigation 123, 3664-3671 (2013)). The poorly formed tumor blood vessels make it difficult to therapeutically increase oxygen delivery to reduce hypoxia (Harrison, D. K. et al. Adv Exp Med Biol 812, 25-31 (2014)). Hypoxia is important clinically because it is a barrier to effective radiation therapy. Molecular oxygen is an electrophile that fixes radiation induced DNA damage (Prise, K. M. et al. Int J Radiat Biol 74, 53-59 (1998) and Johansen, I. et al. Radiation research 24, 184-200 (1965)), enhancing toxicity by 2.5-fold (Freyer, J. P. et al. Radiation research 127, 297-307 (1991)). Reducing tumor hypoxia before radiotherapy should therefore enhance radiation efficacy without increasing the toxicity of well oxygenated normal tissue. In this example, data demonstrating increased tumor oxygenation by decreasing tumor demand for oxygen (Secomb, T. W. et al. Acta Oncol 34, 313-316 (1995)) using FDA-approved drug papaverine is described. Papaverine has an “off target effect” that inhibits mitochondrial complex 1 and oxygen consumption. In vivo, at FDA approved doses, papaverine increases model tumor oxygenation and enhances tumor response to radiation therapy. Genetic studies show that PPV radiosensitization is through inhibition of mitochondrial function. Medicinal chemistry also shows that it is possible to molecularly separated papaverine's classical activity as a PDE10A inhibitor from the newly recognized activity as a mitochondrial inhibitor. PPV derivatives that inhibit complex 1 without inhibiting PDE10A are potential drugs with fewer side effects.

Experimental: Cell lines. All cell lines were purchased from the American Type Culture Collection (ATCC) and grown DMEM (Corning) supplemented with 10% FBS (Seradigm) and 1% Penicillin/Streptomycin (Fisher Bioreagents) at 37° C. in 5% CO2. Cells were treated with inhibitors papaverine hydrochloride (Sigma-Aldrich), rotenone (Sigma-Aldrich), piericidin A (Santa Cruz), capsaicin (Sigma-Aldrich), antimycin A (Sigma-Aldrich) and succinic acid disodium salt (Sigma-Aldrich). Cell viability was assessed by trypan blue exclusion.

CRISPR/Cas9 genetic knockout of NDUFV1. Three separate human NDUFV1 guide RNAs (gRNAs) were obtained from GenSript (catalog no. SC1678, item no. U5053CH250_1-3). Lentiviruses were produced by cotransfection of HEK293T cell line with envelope and packaging vectors (delta 8.2 and VSV G2). Virus-containing media were collected after 48 h and A549 cells were infected in the presence of 8 μg/ml Polybrene (Millipore). After 72 h of selection in 1 μg/ml Puromycin (Sigma-Aldrich) the cells were diluted into single-cell suspensions and individual clones were screened for compromised OCR by Seahorse. NDUFV1 knockout was confirmed by immunoblotting of mitochondrial fraction of candidate A549 clones.

Immunoblotting. Mitochondrial fractions were isolated using mitochondria isolation kit for cultured cells (Thermo Fisher Scientific, catalog no. 89874). 30 g of total mitochondrial protein by BCA was separated on 8% SDS-PAGE gel and then transferred to PVDF membrane. Anti-NDUFV1 (Sigma-Aldrich, SAB2108612), anti-Pyruvate dehydrogenase E1-alpha subunit (Abcam, 110334) and custom anti-NDI1 (gift). Membranes were visualized with IR-fluor labelled secondary antibodies on a Licor scanner.

Seahorse analysis of the OCR. Oxygen consumption rate (OCR) was measured using Seahorse XF96 Flux Analyzer (Agilent Technologies). The cells were seeded to attach overnight, wells were washed with pre-warmed XF Calibrant (Agilent Technologies) and the replaced with unbuffered Assay Medium (pH 7.4, 5 mM glucose, 1 mM L-glutamine). The plates were incubated at 37° C. in a CO₂-free incubator for 2 hours prior to assay. For succinate rescue assay, cells were permeabilized using XF Plasma Membrane Permeabilizer Reagent (XF PMP, Agilent Technologies) according to the manufacturer's instructions. The OCR rate was measured in 1× Mitochondrial Assay Solution (MAS) (70 mM sucrose, 220 mM mannitol, 10 mM KH₂PO₄, 5 mM MgCl₂, 2 mM Hepes, 0.6% (w/v) fatty acid-free BSA and 1 mM EGTA; pH 7.4). After baseline oxygenation was established, 1 μM rotenone or 10 μM PPV was injected at time A, followed by 5 mM succinate at time B. Error bars represent standard deviation. For the washout experiment, EO771 cells were seeded overnight, then treated with 10 μM PPV or 1 μM rotenone 3 hours prior to OCR measurement. Media was replaced 1 and 2 hours before measurement and OCR reported.

In vivo oxygenation measurements. All animal experiments were performed according to protocols approved by institutional IACUC review, with daily veterinarian observation. 2×10⁶ EO771 or 5×10⁶ A549 cells were injected s.c. into the flanks of 6-week old female immunocompromised nu/nu mice. Caliper measurements of opposing diameters were used to calculate the tumor volumes. Upon reaching 500 mm, the animals were anesthetized by inhalation of 1.5% isoflurane and tumor and thigh muscle oxygenation was measured with NIRS optical probe for 30 minutes. Once the baseline oxygenation was established, the animals were injected with either 2 mg per kg of body weight of papaverine hydrochloride or saline by tail vein. Tissue oxygenation was measured for 120 minutes. Obtained oxygenation values were normalized to time of injection and average of 5 traces per treatment averaged. Error bars represent the standard deviation.

Pimonidazole staining. Pimonidazole adducts were visualized in hypoxic regions within histological sections of tumor tissues (Kizaka-Kondoh, S. et al. Cancer science 100, 1366-1373 (2009)). Immune-deficient mice bearing EO771 and A549 flank xenografts were treated with 2 mg/kg PPV or saline and 60 mg/kg pimonidazole administered I.P. at 30 minutes and tumors harvested at 90 minutes. Tumor sections were stained with anti-pimonidazole rabbit antisera and anti-rabbit Alexa Fluor 488 secondary antibody. The hypoxic fraction of each tumor was quantified by thresholding signal at 50% of the maximum signal on control sections. Area covered by pimonidazole-positive cells was evaluated from 20 images per animal and averaged. Error bars represent the standard deviation.

Tumor Growth kinetics. 2×10⁶ EO771 or 5×10⁶ A549 cells were injected s.c. into the flanks of 6-week old female immunocompetent C57/B6 (EO771) or immunocompromised nu/nu (A549) mice. Caliper measurements of opposing diameter were used to calculate the tumor volumes. Upon reaching 150 mm, the tumors were visualized by cone beam CT and treatment plans calculated using SARRP software. X-rays were delivered with a single beam delivering 5 Gy using the Small Animal Research Radiation Platform (SARRP, Xstrahl). Tumor volumes were measured until the post treatment volume increased 3-fold. Error bars represent standard deviation.

TABLE 1
List of cell lines positively tested for sensitivity to PPV

| Cell line | Origin | Species | Cell line | Origin | Species |
|---|---|---|---|---|---|
| EO771 | Breast | Murine | H1568 | Lung | Human |
| HCT116 | Colon | Human | Bone | Normal | Murine |
| | | | marrow | | |
| | | | naïve | | |
| RKO | Colon | Human | HCF | Normal | Human |
| U937 | Leukemia | Human | HEK293T | Normal | Human |
| MOLM13 | Leukemia | Human | OSA16 | Osteosarcoma | Canine |
| TIB-190 | Leukemia | Human | OSA18 | Osteosarcoma | Canine |
| THB-1 | Leukemia | Human | BxPC3 | Pancreas | Human |
| K-562 | Leukemia | Human | Panc1 | Pancreas | Human |
| A549 | Lung | Human | MiaPaca2 | Pancreas | Human |
| HCC-827 | Lung | Human | SU.86 | Pancreas | Human |
| H460 | Lung | Human | PSN-1 | Pancreas | Human |
| H23 | Lung | Human | FaDu | Pharynx | Human |
| H441 | Lung | Human | Ca127 | Tongue | Human |
| H727 | Lung | Human | | | |

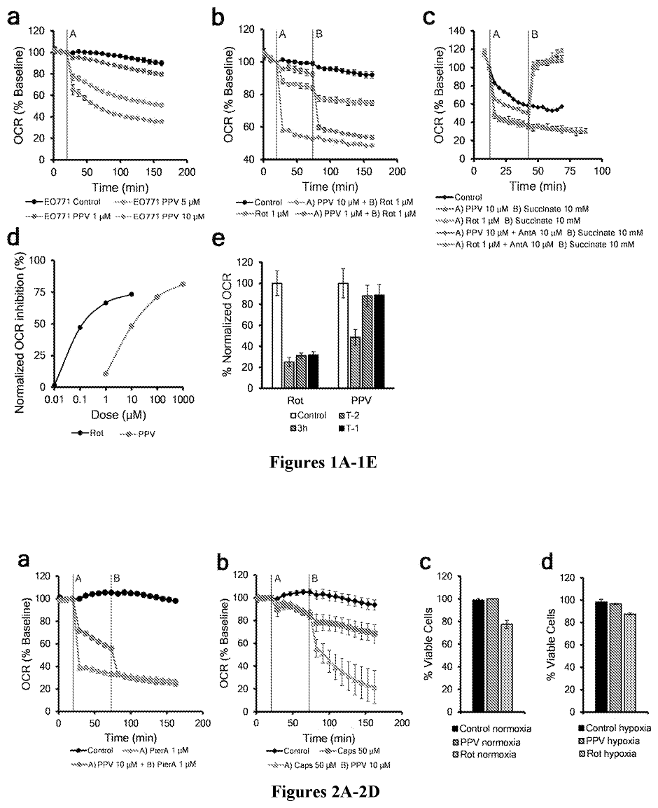

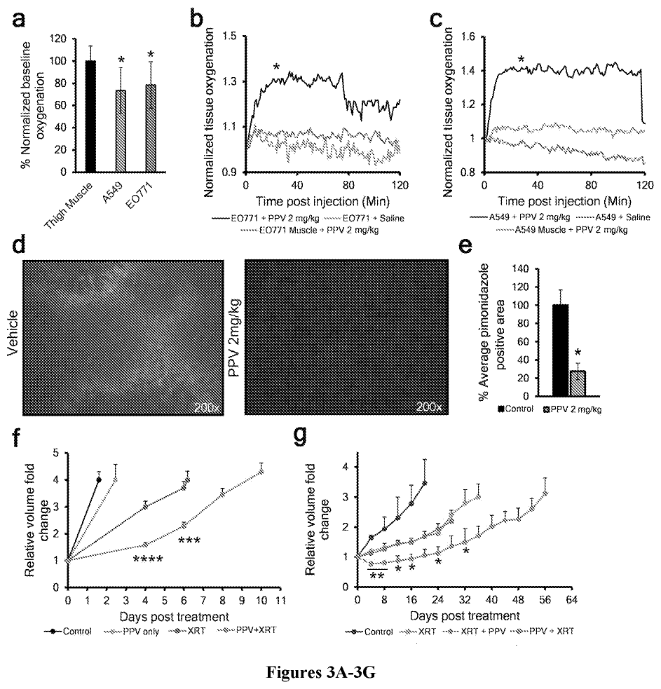

Results: PPV was recently found to slow the growth of cells in media containing only galactose as a carbon source (Gohil, V. M., et al. Nature biotechnology 28, 249-255 (2010)), which indicates inhibition of mitochondrial function. PPV was tested in vitro to determine if it could decrease mitochondrial function in all cells tested in minutes at low micromolar concentrations (Table 1, FIG. 1A). To determine its mechanism of action, PPV in combination with mitochondrial poisons were tested and it was found to block the activity of the classical complex 1 inhibitor rotenone (FIG. 1B), suggesting some competition of the two drugs. In a similar assay with other complex 1 inhibitors piericidin A, or capsaicin there was no interaction (FIGS. 2A-2B). This suggests that PPV may bind to the rotenone site or possibly that its binding blocks this site, in agreement with Morikawa, N. et al. (Journal of neurochemistry 66, 1174-1181 (1996)). To confirm PPV inhibits complex 1, permeabilized cells were treated with either PPV or rotenone followed by complex II substrate succinate that can bypass complex I. FIG. 1C shows that succinate rescued the OCR of both rotenone and PPV-treated cells, but not that of cell treated with the complex 3 inhibitor antimycin A, confirming that PPV action is upstream of complex 2.

Careful dose response analysis shows that that PPV requires 100× higher dose than rotenone for similar OCR inhibition (FIG. 1D). Interestingly, PPV's mitochondrial effect is reversible, in comparison to the more toxic rotenone (Xiong, N., et al. Crit Rev Toxicol 42, 613-632 (2012)). In drug washout experiments baseline OCR returned in less than one hour upon PPV removal, while rotenone treatment showed no restoration in 3 hours, likely explaining papaverine's excellent safety profile (FIG. 1E). De Takats et al. N Engl J Med 282, 225 (1970). In support of its safety, no cellular toxicity in normoxia or hypoxia in vitro was observed (FIGS. 2C-2D).

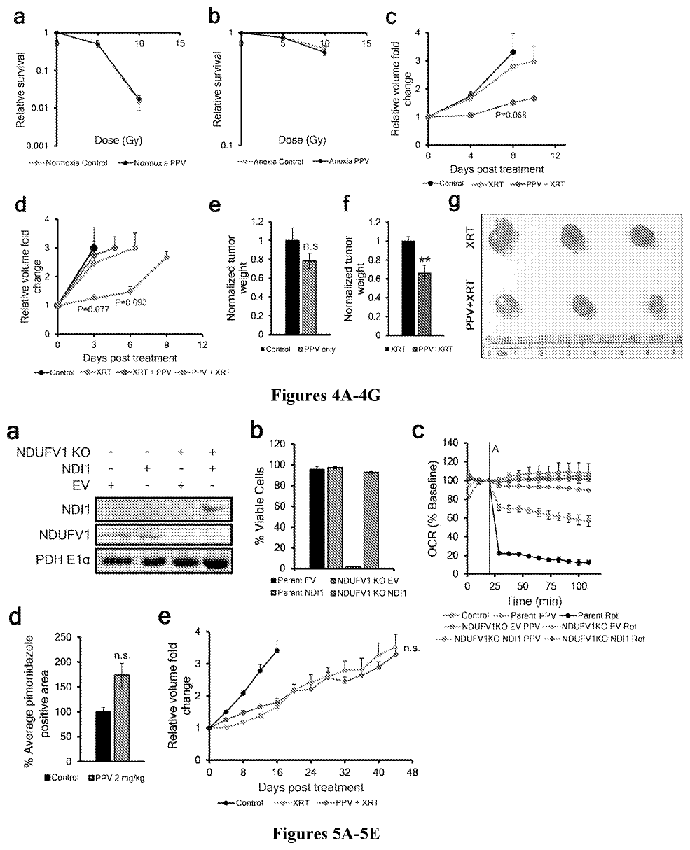

The model described in this example predicts that decreasing oxygen consumption within a tumor will increase overall oxygenation. Therefore, the effect of PPV on oxygenation in transplanted mouse tumors in real time using Fourier domain near infra-red optical spectroscopy (FD-NIRS) was investigated, which averages the oxygenation of the tissue in its light path (Yu, B., et al. J Biophotonics 7, 552-564 (2014)). In heterotopic flank tumors, FD-NIRS shows the baseline oxygenation levels were significantly lower than the same animal's normal thigh muscle (FIG. 3A). After establishing a stable baseline oxygenation, either saline or PPV 2 mg/kg in saline was delivered to the mouse by tail vein. PPV treatment significantly increased the oxygenation of both the breast and lung tumor models within the first 30-45 minutes, while saline treated tumors and PPV treated thigh muscle showed no significant change (FIGS. 3B-3C). In addition, the duration of the effect was consistent with the expected half-life of PPV, between 90-120 min (Ritschel, W. A. et al. Int J Clin Pharmacol Biopharm 15, 227-228 (1977)). These findings were confirmed with the hypoxic marker drug pimonidazole (Rademakers, S. E. et al. BMC Cancer 11, 167 (2011)). PPV treatment for one hour caused a significant 72% decrease in the hypoxic fraction of tumors (FIGS. 3D-3E).

Before testing PPV for radiosensitization of tumors, it was tested in combination with radiation in vitro and found it did not change the surviving fraction of cells irradiated in a dish (FIGS. 4A-4B). Therefore, if potentiation of radiation in vivo was detected, it may be through a secondary effect, like affecting the level of oxygenation. In orthotopic EO771 mammary tumors in mice, a single dose of PPV had no significant effect, but PPV treatment followed by radiation therapy (XRT) produced a 2-4 fold enhancement of tumor growth delay over XRT alone (FIGS. 3F and 4C). Interestingly, in heterotopic A549 lung tumors, PPV treatment before XRT showed a similar effect, but treatment of PPV after XRT did not have any effect compared to radiation alone (FIGS. 3G and 4D). These findings were interpreted to indicate that PPV may affect the tumor metabolism before XRT to achieve radiosensitization.

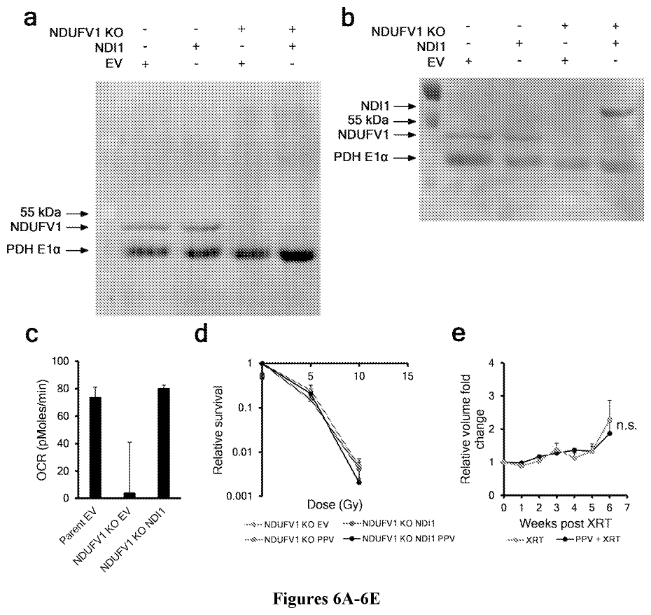

To mechanistically establish that PPV is radiosensitizing through inhibition of mitochondrial complex I, cells with PPV-resistant mitochondria were engineered. CRISPR/Cas9 was used to remove the essential complex 1 subunit NDUFV1 (Benit, P., et al. Am J Hum Genet 68, 1344-1352 (2001)), and then the rotenone-resistant yeast complex 1 paralog NDI1 was introduced into A549 cells (FIGS. 5A and 6A-6B). Previous reports have shown that NDI1 can restore partial mammalian complex I function (Seo, B. B., et al. Proc Natl Acad Sci USA 95, 9167-9171 (1998)). (FIG. 6C). NDI1 was confirmed to rescue mitochondrial function by showing it could support cell survival in media with only galactose as an energy source (FIG. 5B). Further analysis of these cells by Seahorse revealed that their mitochondria were resistant to both PPV and rotenone (FIG. 5C). Cells with papaverine-resistant mitochondria were then used to grow tumors in mice to test for PPV-dependent effects in vivo. Tumor bearing mice were treated with PPV, followed by pimonidazole. Staining of sections showed no decrease of the hypoxic fraction (FIG. 5D). A second set of tumor-bearing animals were treated with either radiation or PPV followed by radiation. FIG. 5E shows these tumors are resistant to PPV dependent increase in radiation growth delay, confirming that PPV radiosensitizes tumors through inhibition of complex I (FIGS. 5E and 6E).

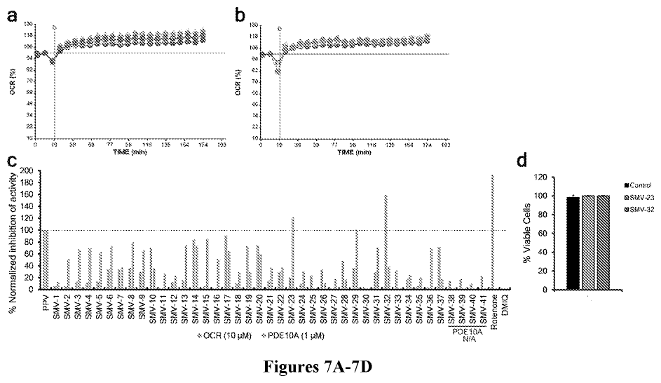

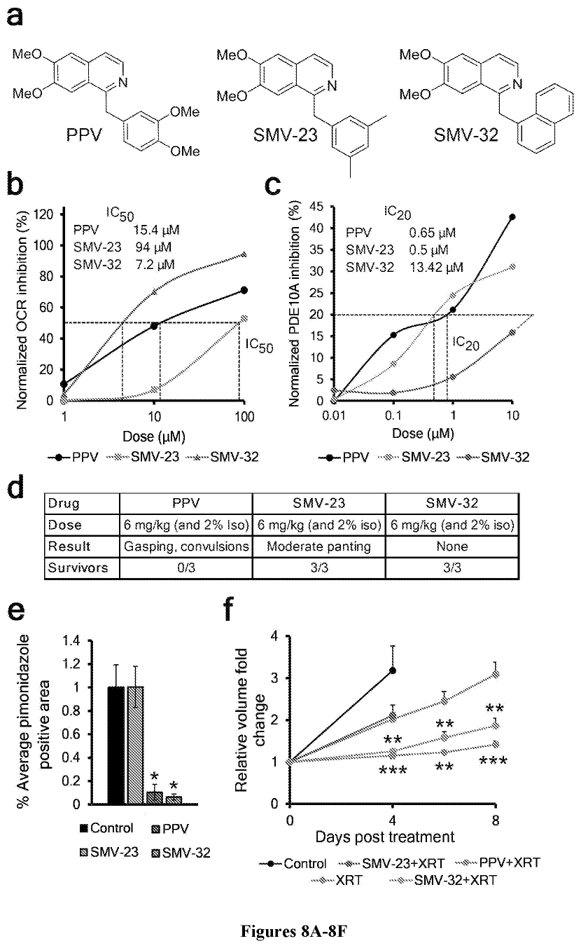

PPV has a long history of clinical use as a treatment of cerebral and peripheral arterial spasm due to its activity as a phosphodiesterase 10A (PDE10A) inhibitor (Gagnon, G. et al. Br J Pharmacol 70, 219-227 (1980)). However, in vitro treatment of cells with other PDE inhibitors or 8-Br-cAMP showed no decrease in OCR, indicating that elevated cAMP was not responsible for its mitochondrial effects (FIGS. 7A-7B). Therefore, the PPV molecule was re-engineered to separate the PDE activity from the mitochondrial activity. These derivatives could support the model of PPV radiosensitization and remove unwanted activity that might result in potential side effects. Analysis of 41 lead derivatives of PPV identified lead compounds that have separated the OCR and PDE inhibition activity by over 10-fold in vitro (Table 2, FIGS. 7C and 8A-8C). By Seahorse analysis and PDE10A enzymes assay, SMV-23 has a mitochondrial IC50 of 94 uM and PDE IC20 of 0.5 uM while SMV-32 has a mitochondrial IC₅₀ of 7.4 uM and PDE IC20 of 12 uM (FIGS. 8B-8C). Neither of these compounds shows cellular toxicity at 10 uM in vitro (FIG. 7D), the safety of these compounds compared to the parent molecule was determined. PPV has a high rodent LD50 when delivered slowly, but it can be toxic when delivered quickly because it acutely decreases vascular compliance leading to cardiovascular collapse. In isoflurane-anesthetized mice a rapid dose of 6 mg/kg PPV was found to be toxic. However, neither of the two lead derivatives showed toxicity at this dose, suggesting that the combination of PDE and mitochondrial activities resulted in PPV toxicity (FIG. 8D). In vivo, SMV-32 decreased tumor hypoxia by pimonidazole staining while SMV-23 had no effect on hypoxia (FIG. 8E). Finally, these molecules were compared to the parent molecule for radiosensitization of tumors. In heterotopic EO771 tumors, SMV-23 showed no increase in growth delay when compared to radiation alone, while SMV-32 shows activity that is comparable to papaverine (FIG. 8F).

TABLE 2
Inhibitory activity of compounds described herein.

| | | Inhibitory activity | |
|---|---|---|---|
| | | | PDE10A |
| | MW | OCR | % of |
| Compound No. | (g/mol) | % of PPV | PPV |
| 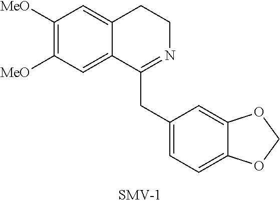 [SMILES: COc1cc2c(cc1OC)C(Cc1ccc3c(c1)OCO3)=NCC2] | 325.36 | 5.55 | 12.77 |
| 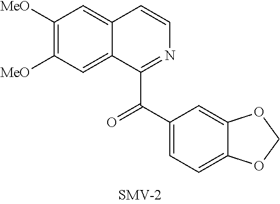 [SMILES: COc1cc2ccnc(C(=O)c3ccc4c(c3)OCO4)c2cc1OC] | 337.33 | 5.55 | 51.12 |
| 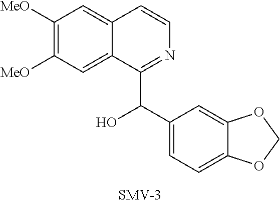 [SMILES: COc1cc2ccnc(C(O)c3ccc4c(c3)OCO4)c2cc1OC] | 339.35 | 12.96 | 67.90 |
| 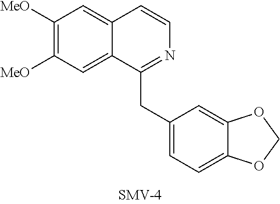 [SMILES: COc1cc2ccnc(Cc3ccc4c(c3)OCO4)c2cc1OC] | 323.35 | 11.11 | 69.54 |
| 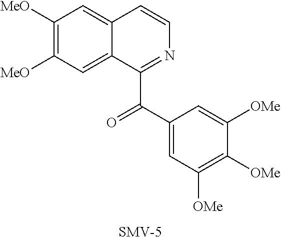 [SMILES: COc1cc2ccnc(C(=O)c3cc(OC)c(OC)c(OC)c3)c2cc1OC] | 383.40 | 13.63 | 63.39 |
| 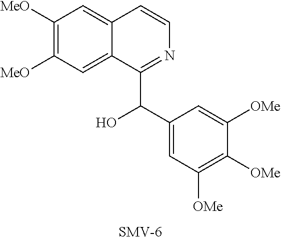 [SMILES: COc1cc2ccnc(C(O)c3cc(OC)c(OC)c(OC)c3)c2cc1OC] | 385.42 | 34.09 | 72.86 |
| 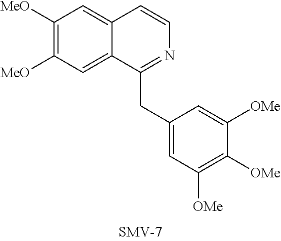 [SMILES: COc1cc2ccnc(Cc3cc(OC)c(OC)c(OC)c3)c2cc1OC] | 369.42 | 34.09 | 37.37 |
| 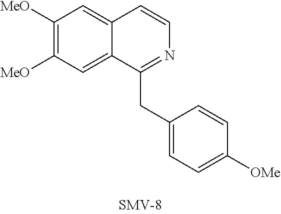 [SMILES: COc1ccc(Cc2nccc3cc(OC)c(OC)cc23)cc1] | 309.37 | 36.36 | 79.93 |
| 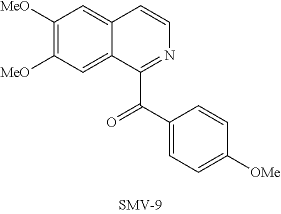 [SMILES: COc1ccc(C(=O)c2nccc3cc(OC)c(OC)cc23)cc1] | 323.35 | 29.55 | 65.88 |
| 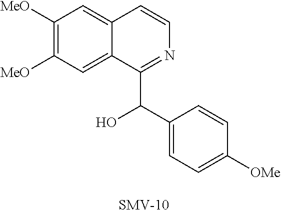 [SMILES: COc1ccc(C(O)c2nccc3cc(OC)c(OC)cc23)cc1] | 325.36 | 70.45 | 35.44 |
| 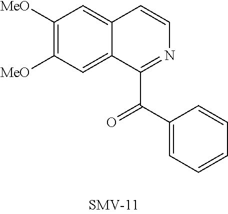 [SMILES: COc1cc2ccnc(C(=O)c3ccccc3)c2cc1OC] | 293.32 | 0 | 26.28 |
| 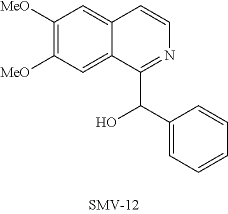 [SMILES: COc1cc2ccnc(C(O)c3ccccc3)c2cc1OC] | 295.34 | 12.07 | 23.60 |
| 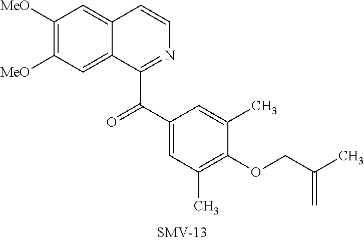 [SMILES: C=C(C)COc1c(C)cc(C(=O)c2nccc3cc(OC)c(OC)cc23)cc1C] | 391.47 | 15.51 | 74.38 |
| 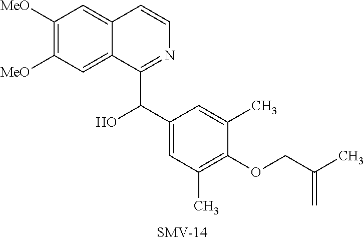 [SMILES: C=C(C)COc1c(C)cc(C(O)c2nccc3cc(OC)c(OC)cc23)cc1C] | 393.48 | 84.42 | 73.33 |
| 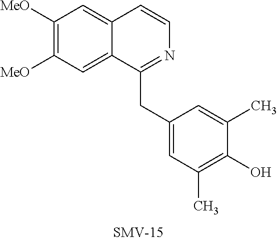 [SMILES: COc1cc2ccnc(Cc3cc(C)c(O)c(C)c3)c2cc1OC] | 323.39 | 5.86 | 85.02 |
| 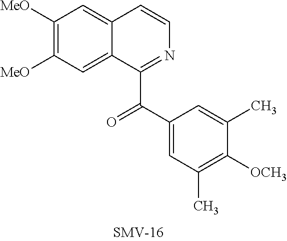 [SMILES: COc1cc2ccnc(C(=O)c3cc(C)c(OC)c(C)c3)c2cc1OC] | 351.40 | 0 | 50.92 |
| 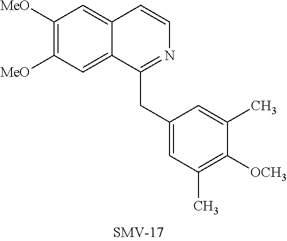 [SMILES: COc1cc2ccnc(Cc3cc(C)c(OC)c(C)c3)c2cc1OC] | 337.42 | 91.18 | 64.65 |
| 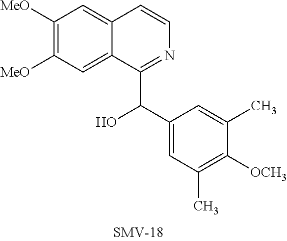 [SMILES: COc1cc2ccnc(C(O)c3cc(C)c(OC)c(C)c3)c2cc1OC] | 353.42 | 10.35 | 29.18 |
| 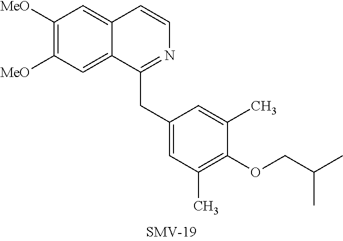 [SMILES: COc1cc2ccnc(Cc3cc(C)c(OCC(C)C)c(C)c3)c2cc1OC] | 379.50 | 72.88 | 29.13 |
| 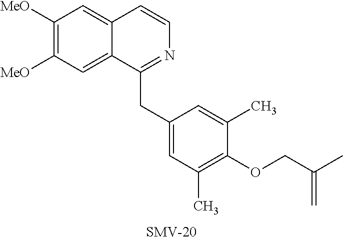 [SMILES: C=C(C)COc1c(C)cc(Cc2nccc3cc(OC)c(OC)cc23)cc1C] | 377.48 | 74.81 | 59.69 |
| 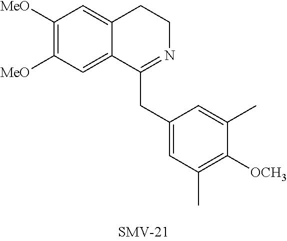 [SMILES: COc1cc2c(cc1OC)C(Cc1cc(C)c(OC)c(C)c1)=NCC2] | 339.44 | 14.81 | 37.27 |
| 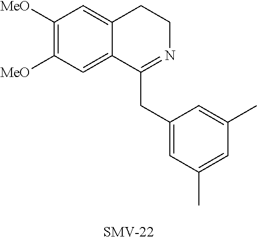 [SMILES: COc1cc2c(cc1OC)C(Cc1cc(C)cc(C)c1)=NCC2] | 309.41 | 30.09 | 40.00 |
| 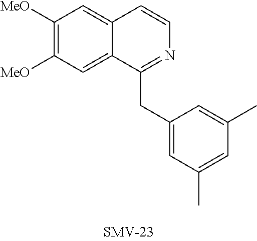 [SMILES: COc1cc2ccnc(Cc3cc(C)cc(C)c3)c2cc1OC] | 307.39 | 20.37 | 121.49 |
| 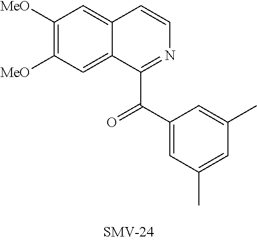 [SMILES: COc1cc2ccnc(C(=O)c3cc(C)cc(C)c3)c2cc1OC] | 321.38 | 9.26 | 30.52 |
| 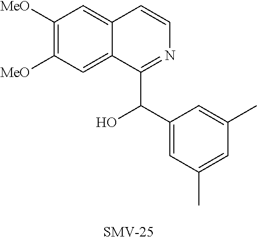 [SMILES: COc1cc2ccnc(C(O)c3cc(C)cc(C)c3)c2cc1OC] | 323.39 | 23.40 | 0 |
| 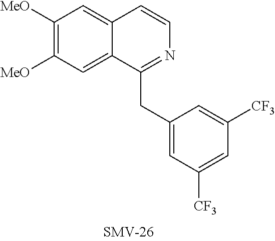 [SMILES: COc1cc2ccnc(Cc3cc(C(F)(F)F)cc(C(F)(F)F)c3)c2cc1OC] | 415.34 | 33.4 | 10.28 |
| 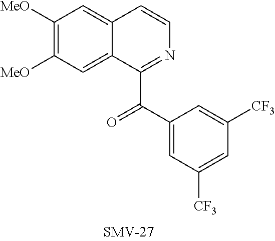 [SMILES: COc1cc2ccnc(C(=O)c3cc(C(F)(F)F)cc(C(F)(F)F)c3)c2cc1OC] | 429.32 | 0 | 17.69 |
| 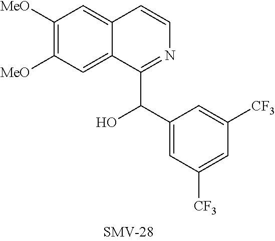 [SMILES: COc1cc2ccnc(C(O)c3cc(C(F)(F)F)cc(C(F)(F)F)c3)c2cc1OC] | 431.33 | 48.27 | 16.58 |
| 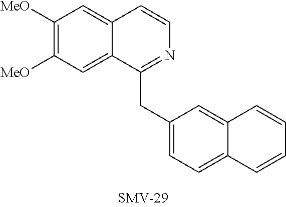 [SMILES: COc1cc2ccnc(Cc3ccc4ccccc4c3)c2cc1OC] | 329.40 | 35.99 | 100 |
| 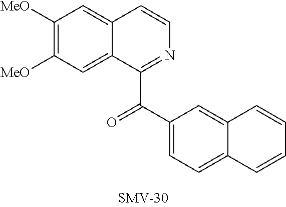 [SMILES: COc1cc2ccnc(C(=O)c3ccc4ccccc4c3)c2cc1OC] | 343.38 | 4.34 | 0 |
| 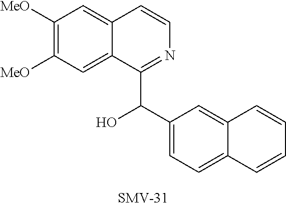 [SMILES: COc1cc2ccnc(C(O)c3ccc4ccccc4c3)c2cc1OC] | 345.40 | 29.26 | 70.84 |
| 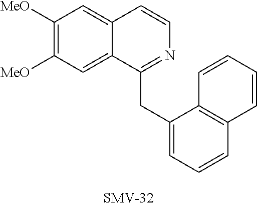 [SMILES: COc1cc2ccnc(Cc3cccc4ccccc34)c2cc1OC] | 329.40 | 159.3 | 38.66 |
| 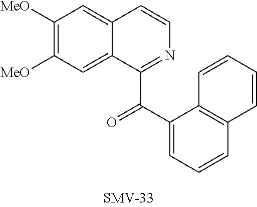 [SMILES: COc1cc2ccnc(C(=O)c3cccc4ccccc34)c2cc1OC] | 343.38 | 31.91 | 0 |
| 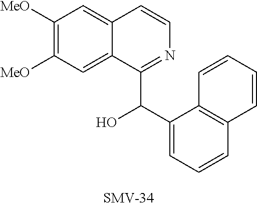 [SMILES: COc1cc2ccnc(C(O)c3cccc4ccccc34)c2cc1OC] | 345.40 | 18 | 24.24 |
| 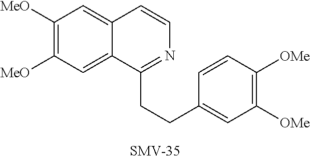 [SMILES: COc1ccc(CCc2nccc3cc(OC)c(OC)cc23)cc1OC] | 353.42 | 5.94 | 20.16 |
| 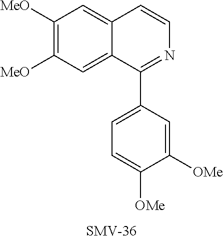 [SMILES: COc1ccc(-c2nccc3cc(OC)c(OC)cc23)cc1OC] | 325.36 | 3.64 | 69.55 |
| 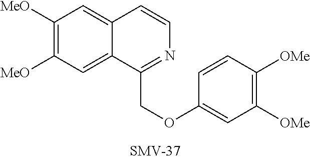 [SMILES: COc1ccc(OCc2nccc3cc(OC)c(OC)cc23)cc1OC] | 355.39 | 72.05 | 17.18 |
| 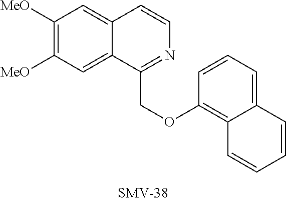 [SMILES: COc1cc2ccnc(COc3cccc4ccccc34)c2cc1OC] | 345.40 | 14.19 | N/A |
| 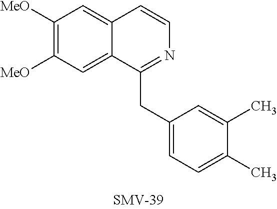 [SMILES: COc1cc2ccnc(Cc3ccc(C)c(C)c3)c2cc1OC] | 307.49 | 17.43 | N/A |
| 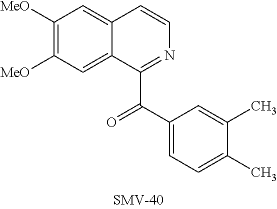 [SMILES: COc1cc2ccnc(C(=O)c3ccc(C)c(C)c3)c2cc1OC] | 321.38 | 9.64 | N/A |
| 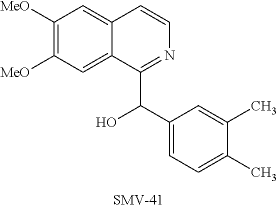 [SMILES: COc1cc2ccnc(C(O)c3ccc(C)c(C)c3)c2cc1OC] | 323.39 | 22.69 | N/A |
| 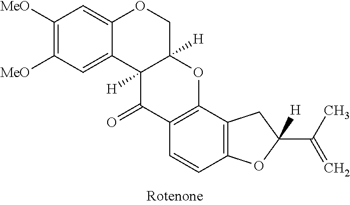 [SMILES: C=C(C)[C@H]1Cc2c(ccc3c2O[C@@H]2COc4cc(OC)c(OC)cc4[C@@H]2C3=O)O1] | 394.41 | 193 | 0 |
| 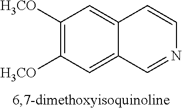 [SMILES: COc1cc2ccncc2cc1OC] | 189.21 | 0 | 0 |
| DMIQ | | | |

Summary: This example shows that a single dose of an FDA-approved drug inhibits mitochondrial function and transiently reduces tumor hypoxia providing a clinically-manageable therapeutic window to deliver more effective radiation therapy. Papaverine or one of its derivatives appears to be ideal candidates for this approach because it overcomes drawbacks to other molecules proposed as metabolic radiosensitizers. PPV is safe, cell permeable, rapid, reversible, and quickly cleared from the patient (Heinz, S., et al. Sci Rep 7, 45465 (2017); Chandel, N. S., et al. Cell metabolism 23, 569-570 (2016); Ashton, T. M., et al. Nat Commun 7, 12308 (2016); and Ellinghaus, P., et al. Cancer medicine 2, 611-624 (2013)). The key to the use of such a radiosensitizer will be identifying those tumors with extensive hypoxia that would be predicted to benefit from this approach.

### Example 2

Tumor hypoxia remains a barrier to effective radiation therapy: It has been recognized for over 60 years that hypoxia protects organisms from the detrimental effects of ionizing radiation. The “oxygen enhancement ratio” or OER is approximately 2.5-3 fold. This means it takes 2.5-3 times the dose of radiation delivered to very hypoxic cells to get the same amount of cell kill if those cells had been fully oxygenated. Biophysical analysis supports a model where oxygen acts as an electrophile to fix DNA damage within nanoseconds of radiation delivery, lack of fixation allows for resolution of metastable DNA radical intermediates in hypoxia.

Even though tumor hypoxia has been studied preclinically, clinical approaches designed to overcome hypoxia have disappointing results. Strategies designed to deliver more oxygen to the tumor, deliver oxygen-mimetics, or deliver drugs with preferential toxicity towards hypoxic cells have all been effective in rodent studies but failed in human trials. The most widely studied strategy has been to increase delivery of oxygen to tumors. However, this is limited by the inherent nature of the poorly formed and poorly functioning tumor vascular tree. Vessels in transplanted and spontaneous tumors have blind ends, breaks, and tortuous paths, all of which reduce laminar flow and decrease oxygen delivery.

Because tumor hypoxia is an inherent mismatch between supply and demand, described herein is an alternative approach to address tumor hypoxia by decreasing the demand side rather than increasing the supply side. Mitochondria are the major sink for molecular oxygen in the cell, so mitochondrial inhibitors that could reduce oxygen consumption in hypoxic tissue and bring oxygen supply and demand into balance was investigated. In order to make such a strategy translatable, FDA approved drugs for those with off target effects on the mitochondria (NCI P01 CA016676) was investigated. The molecule with the best characteristics was papaverine, an ergot alkaloid first isolated in 1848 and subsequently used as a smooth muscle relaxant (preliminary data).

Papaverine as a Mitochondrial Inhibitor Reduces Tumor Hypoxia: Papaverine (PPV) is FDA approved and had been used as a smooth muscle relaxant for the treatment of vasospasm, angina, and erectile dysfunction. It is thought to act as a phosphodiesterase 10A inhibitor, with a mechanism of action similar to sildenafil and tadalafil. Papaverine is cell permeable and reversibly inhibits mitochondrial function in minutes in the low micromolar concentration in vitro. PPV is also rapidly cleared from the serum with a 1.5-2 h half-life so its effects are rapid, reversible and significant at doses approved for human use (1 mg/kg, FDA website). The anti-mitochondrial activity of PPV as working through an inhibition of electron transport chain (ETC) complex 1 has also been clarified. Data presented in this example show PPV can compete with the classical complex 1 inhibitor rotenone at the rotenone binding site on complex 1. Data also shows that systemic delivery of PPV can increase oxygenation in transplanted tumors grown in mice, and this increase in oxygenation results in a radiosensitization that increases tumor growth delay by 2-4 fold. The rapid, reversible and sensitive ability of PPV to inhibit mitochondrial oxygen consumption in vivo make it an ideal candidate as an effective hypoxic tumor radiosensitizer that could be used in humans in treatment protocols involving radiation.

Clinical identification of hypoxic tumors: Ever since it was recognized that tumor hypoxia was a modifier of radiotherapy, significant effort has been invested in the search for an easy, non-invasive method to identify hypoxic tumors. The state of the art solutions to this problem fall into 2 main categories: 2-nitroimidozole marker drugs that bind in hypoxic tissue, and biological indicators of hypoxic response such as hypoxia-inducible genes. The nitroimidazoles like misonidazole, pimonidazole or EF5 are given systemically and are substrates for cellular nitroreductases. The drugs are metabolized to the radical form and in hypoxic tissue they form covalent adducts to cellular macromolecules. When oxygen is present, the drugs are oxidized back to the parent molecule. The remaining adducts can be detected by PET imaging if the molecules are labelled with a positron emitter, or by immunohistochemistry with specific monoclonal antibodies if a biopsy is available. Histochemical analysis is cheaper, less time-dependent and perhaps more reproducible. Although the benefit of the PET tracer is that the whole tumor is imaged, the dynamic range of the PET tracers is somewhat limited. Expression of hypoxia-inducible genes and proteins can also be used, but somewhat difficult to quantitate due to oxygen level variations, other micro-environmental stresses, and kinetics of gene induction and turnover.

Frequency-domain near-infrared spectroscopy (FD-NIRS) can noninvasively quantify tissue hemoglobin concentration (THb) and oxygenation (SO₂) in real time. This technology uses light in the 700-800 nm wavelength because it has reasonably good tissue penetration (nominally 1-2 cm in depth). A FD-NIRS system with a novel side firing probe to measure changes in tumor oxygenation in response to pharmacological intervention was developed and used in this example. This example has generated in vivo data showing that papaverine can increase oxygenation in xenografted tumors in mice.

The FD-NIRS instrument consists of a network analyzer as the radio frequency (RF) source and phase-sensitive detection system, a 1×4 RF switch, a 4×2 optical switch, a multi-channel laser diode controller, four laser diodes (654, 779, 805 and 847 nm) installed in mounts with a bias-T, an avalanche photodiode (APD) module, and a laptop computer loaded with custom software. The surface probe consists of two side-firing source fibers (200/220/240 □m, NA=0.22) and a single side-firing detection fiber (600/660/710 □m, NA=0.22), providing two source-detector separations (SDSs) of 5 and 10 mm, respectively. The fiber tips are polished to a 450 angle and coated with a mirror to create a light path perpendicular to the fiber. All the fiber tips are epoxied into a V-groove on a thin Delrin plate (biocompatible) so that it can be easily attached to the skin surface above a tumor. The two SDSs allow elimination of artifacts due to instrument response and the skin effects, while providing a penetration depth around 10-15 mm in typical tissue.

The FD-NIRS instrument launches intensity-modulated continuous wave lasers into the tissue and collects the amplitude-attenuated and phase-shifted diffuse reflectance from the tissue at the same frequency of the incident light, both through the side-firing probe. The optical switch outputs each of the four wavelengths to one of the two source fibers sequentially and the modulation frequency is scanned from 50 to 450 MHz at a 2 MHz interval at each wavelength. A custom LabVIEW program integrated with MATLAB scripts is used for instrument control, data acquisition, and data analysis and display. The measured tissue total hemoglobin content (THb) and oxygenation (SO₂) is displayed on the computer screen in real-time.

Results:

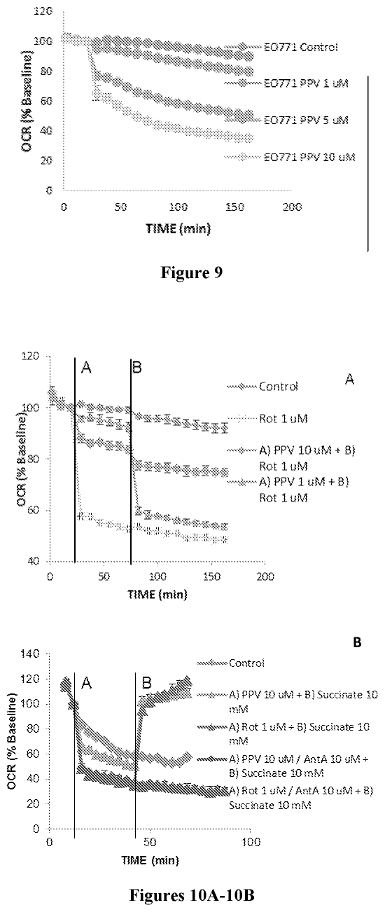

Papaverine is an effective mitochondrial electron transport chain (ETC) complex 1 antagonist: In the current example, compounds that can decrease tumor hypoxia by decreasing oxygen consumption within the tumor cells were identified. Metformin, phenformin, meclizine, ranolazine, and papaverine as effective at inhibiting oxygen consumption in vitro by Seahorse XF analysis were identified. Penetration into the cell (metformin), dose required for significant inhibition (meclizine), and maximal activity (ranolazine) disqualified some compounds for use as radiosensitizers. Papaverine, however, rapidly inhibited oxygen consumption at low micromolar concentrations that are achievable with established clinical doses. PPV is cell penetrable and its effectiveness in murine was tested in vitro (FIG. 9).

The activity of PPV as a smooth muscle relaxant is attributed to inhibition of phosphodiesterase (PDE) 10A. Classical PDE inhibitors as well as 8-bromocAMP were investigate and none was found to inhibit mitochondrial function in vitro. PPV's mitochondrial activity may be through an unrecognized “off target” mechanism. PPV action in combination with classical cytochrome oxidase (KCN), complex 3 (antimycin A), and complex 1 (rotenone) inhibitors was investigated. There was no effect on the activity of downstream inhibitors, but an interaction with rotenone. FIG. 10A shows that papaverine can interfere with the inhibitory activity of rotenone at complex 1. Addition of papaverine before rotenone blocked rotenone's inhibitory activity, suggesting a competition of the two drugs for inhibition of complex 1. In depth analysis of other complex 1 inhibitors showed that papaverine specifically interferes with rotenone, presumably through competition at the rotenone binding pocket (at approximately 100× lower efficiency). To confirm that PPV was acting as a complex 1 inhibitor, studies if complex 2 substrates could rescue OCR in cells treated with PPV, bypassing the block to electron transport were conducted. FIG. 10B shows that in permeabilized cells, addition of succinate can recuse OCR in cells treated with rotenone or PPV, but not in cells treated with the complex three inhibitor antimycin A. To better understand the clinical utility of PPV, the reversibility of PPV activity was investigated and found that PPV OCR changes could be efficiently washed out within one hour of changing the media, as opposed to rotenone that is an irreversible inhibitor.

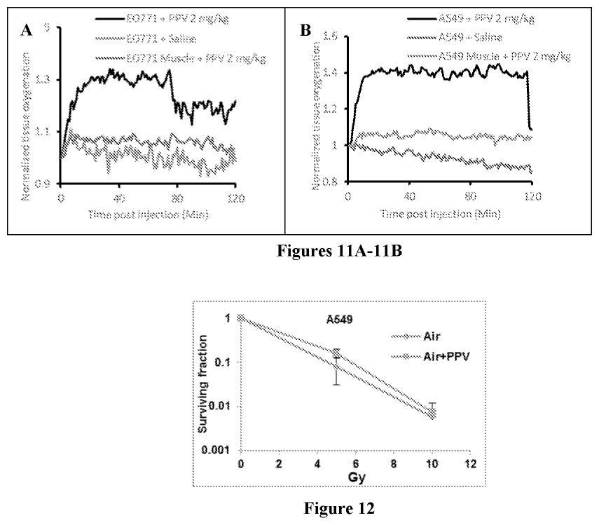

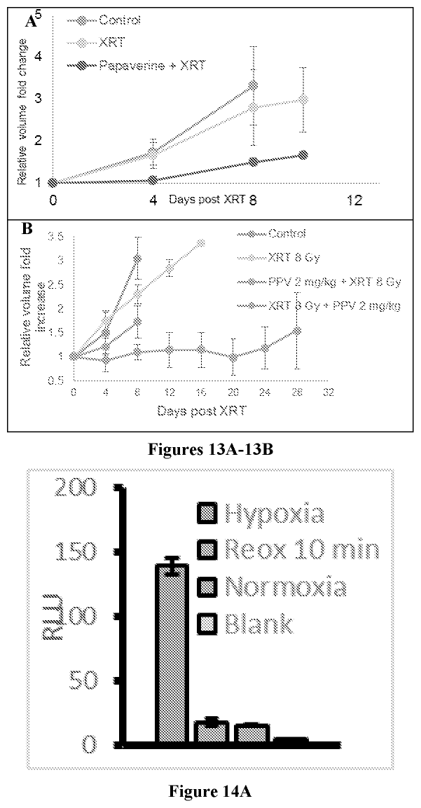

Papaverine can increase oxygenation in model rodent tumors: In vitro analysis of PPV indicated that it fit the criteria of a potential radiosensitizer through reduction in tumor hypoxia: its effect is rapid, active at clinically achievable doses, reproducible in a variety of cells, and rapidly reversible (reduces the likelihood of adverse effects after radiation is given). PPV's ability to increase oxygenation in model tumors using the FD-NIRS was investigated. This optical probe could detect changes in oxygenation in real time, over a matter of minutes. Transplanted flank tumors were grown in nude mice from either the human A549 lung adenocarcinoma cells, or the murine EO771 breast cancer cells. When the tumors reached approximately 1 cm³ in volume, they were large enough for the side firing probe. Tumor bearing mice were anesthetized, the probe was placed on the tumor (or normal thigh muscle) and measurements taken continuously every 15 seconds. After 20 minutes to establish a baseline, 2 mg/kg papaverine in saline was injected i.v. and the tumor oxygenation followed. FIG. 11 shows that in both model tumors, oxygenation rose dramatically to a maximum 1.35-1.45× above baseline 30 min post injection. Some decease was detected by 100 minutes, and no change was detected in tumors of mice injected with saline, or in the normal thigh muscle of papaverine treated mice.

Papaverine is an effective radiosensitizer of subcutaneous and orthotopic model tumors: Because papaverine could significantly increase oxygenation in transplanted rodent tumors, its ability to act as a radiosensitizer through its activity as a metabolic modifier was investigated. This is an effect that was referred to as metabolic radiosensitization because the drug alters tumor oxygen metabolism to achieve radiosensitization. To ensure that any radiosensitization was not through another unrecognized activity, papaverine was tested for capacity as an inherent radiosensitizer. Treatment of A549 cells in vitro with 5 μM papaverine for one hour prior to radiation did not have any effect on colony survival in vitro (FIG. 12). These results indicate that if papaverine has an effect as a radiosensitizer in vivo, that it would necessarily have to be through a secondary effect, such as reduction in tumor hypoxia.

Both models of transplanted tumors that were used for the oxygenation experiments of papaverine's effect on radiation response in vivo were investigated. The murine breast cancer line EO771 that is syngeneic with the BL6 strain of mice was used and transplanted it orthotopically into the mammary fat pad of immune-competent mice. Also used was the human A549 lung adenocarcinoma line that was transplanted into the flank of immune-deficient mice. The breast cancers grew to approximately 150 mm³ and were then randomized to receive either no treatment, a single dose of either 5 Gy, or papaverine at 2 mg/kg followed 30-40 minutes later by 5 Gy. Tumors were imaged by cone beam CT and irradiated with a single tangential beam on the small animal radiation research platform (SARRP) to achieve >95% of the prescribed dose to the entire tumor. FIG. 13A shows that addition of papaverine significantly enhanced the antitumor effects of the radiation in the orthotopic, immune-competent breast model (P<0.05).

For the test of the A549 flank tumors, the tumors were treated similarly to the breast cancer model, but 8 Gy was used, and a treatment group added consisting of 8 Gy radiotherapy followed 30 minutes later by 2 mg/kg papaverine. The reverse schedule would indicate if the drug must be present before the radiation so that it would alter the tumor metabolism to make it more susceptible to the radiotherapy. If papaverine was inherently toxic on its own for some reason, then the order it is given with radiation would not alter its growth inhibiting properties. Tumors were imaged with cone beam CT and irradiated using the small animal radiation research platform (SARRP) using a single tangential beam giving greater than 95% of the tumor the prescribed dose.

FIG. 13 shows that for either the orthotopic EO771 or the flank A549 tumors that the addition of papaverine before the radiation resulted in a significant enhancement of the growth delay. For the EO771 tumors, the growth delay in this and in additional experiments were estimated to be approximately 1.9-2.8 fold greater with papaverine (the experiment shown in FIG. 12A was stopped early due to ulceration of skin over the tumors by day 10 post treatment). In the A549 flank tumor experiments, the addition of papaverine increased growth delay by approximately 2.1-3.9 fold. These and other experiments show that in model tumors grown in mice, addition of 2 mg/ml papaverine 30-60 minutes prior to radiation increased relative growth delay of a single dose of radiation by 1.9-3.9 fold.

Papaverine's dual activities are separable. Papaverine has been traditionally thought to be effective as a vasodilator for the treatment of erectile dysfunction because it can inhibit phosphodiesterase 10A (similar to PDE 5 inhibitors sildenafil and tadalafil). However, tadalafil and 8-bromo-cAMP were tested for effects on OCR and they were found to be ineffective.

Because PPV's effect on mitochondrial function is independent of cAMP levels it was reasoned that there are two activities in PPV, one inhibiting complex 1 and one inhibiting PDE10A that may reside in different parts of the molecule. The structure activity relationship of PPV relative to two assays was assessed: inhibition of cellular OCR by Seahorse, and inhibition of PDE10A by purified enzyme activity in multiwell plate (PDE10A activity assay kit, BPS Bioscience). Because PPV can compete with rotenone as complex 1 inhibitors, it was reasoned that they may be binding the same site and therefore the structures would be similar relative to the mitochondrial binding target. It was found that the top half of the PPV molecule (DMIQ) was inactive in either assay, indicating both top and bottom were necessary (FIG. 7C). Because the top was a planar structure similar to that of rotenone, and they compete with each other for the same binding pocket on complex 1 (FIG. 10) the lower portion of the molecule and how it may resemble rotenone were investigated in more detail. It was established that flexible linkage was preferred for mitochondrial activity and bulky adducts to the lower benzene ring may block it (FIG. 7C). Following this strategy, analysis of the first 37 compounds show that a “relatively pure” complex 1 inhibitor and a “relatively pure” PDE10A inhibitor were identified. When compared to PPV, SMV23 shows 125% PDE10 activity and 20% complex 1 activity while SMV 32 shows 160% complex 1 activity and 40% PDE10A activity. SMV32 complex 1 activity is approaching that of rotenone. These lead compounds were synthesized from commercial precursors and purified on silica gel column. Structures were confirmed by ¹H NMR and found to be >95% pure by HPLC.

These two compounds can be synthesized at gram quantities and be used for in vivo experiments. They are relatively soluble in aqueous solutions and can now be used to test the hypothesis that the radiosensitizing effect of PPV stems from its ability to inhibit mitochondrial function, not PDE10A.

Reducing any possible adverse effects of PPV given to cancer patients if it is being used as a radiosensitizer are needed. PDE10A inhibition can cause systemic vascular effects which can result in a drop in blood pressure and increase in heart rate as vessels become more compliant. This acute drop in blood pressure produces a physiological compensation where the individual's heart rate and breathing rate rise to maintain tissue perfusion. If the effect on systemic vasculature was too great, this could lead to cardiovascular collapse and rapid death. Acute toxicity of PPV, SMV23 and SMV32 given IV to mice under anesthesia with 2% isoflurane was determined. Isoflurane delivered in medical air was used so that the effects on tumor oxygenation was reduced, although isoflurane can depress respiration and is typically given in 100% oxygen. If either drug is given as one large bolus in less than a minute, then all are tolerated up to 4 mg/kg. At 6 mg/kg, PPV becomes toxic, with significant gasping of mice, convulsions and death within 5 minutes of delivery. At 6 mg/kg SMV23 causes some moderate panting, but not toxicity, and mice recover within 10 minutes. At 6 mg/kg SMV32 shows no obvious signs of distress (Table 3). These results suggest that we have been successful in the separation of the vascular effects of PPV from its activity as a mitochondrial inhibitor. The toxicity of PPV at high doses stems from the combined effects of both activities. Separating these activities in SMV23 and SMV32 makes both derivatives tolerable at high doses. SMV32 therefore could be a superior agent as a radiosensitizer when compared to the parent PPV molecule, especially for patients with cardiovascular comorbidities. We therefore propose to continue development of PPV as a metabolic radiosensitizer (in pending NIH applications) as well as our new molecule that we feel has the potential for superior results, especially with the reduction of possible cardiovascular side effects.

TABLE 3
Acute toxicity of papaverine and derivatives given IV to
mice anesthetized with 2% isoflurane in medical air.
Note, if drug is given 2 mg/kg every 10 minutes, then all
drugs are tolerated at 12 mg/kg total dose (over 60 minutes).

| Drug | PPV | SMV23 | SMV 32 |
|---|---|---|---|
| Dose | 6 mg/kg (and | 6 mg/kg (and | 6 mg/kg (and |
| | 2% Iso) | 2% iso) | 2% iso) |
| Result | Gasping, | Moderate | None |
| | convulsions | panting | |
| Survivors | 0/3 | 3/3 | 3/3 |

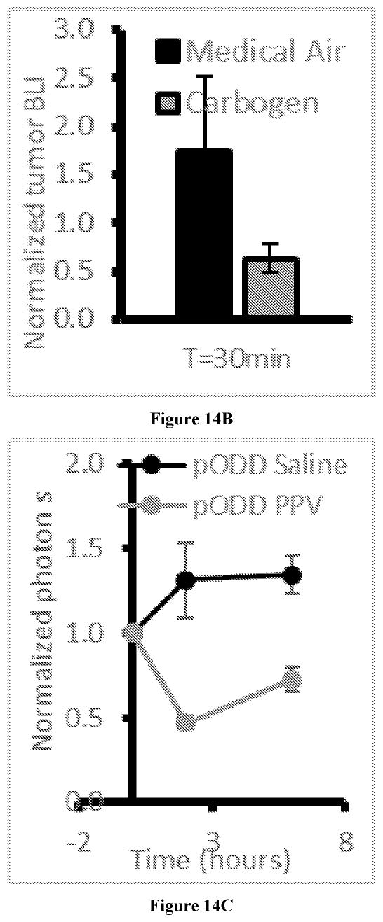

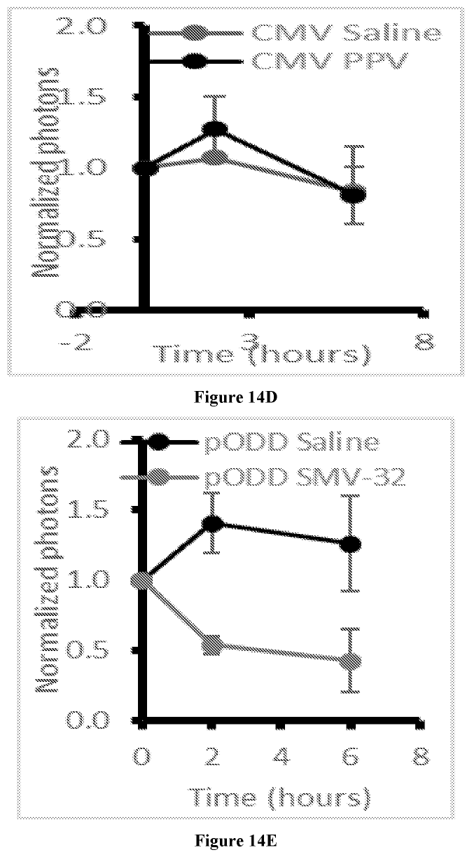

Use of ODD-luciferase as an in vivo oxygen reporter. We have fused the oxygen dependent degradation domain (ODD) of the HIF1a protein to the luciferase reporter protein. This fusion protein is expressed in tumor cells and is stable and reports well in hypoxia, but is destroyed by cellular machinery when cells are well oxygenated (FIG. 14A). These cells were used to grow tumors in immune-deficient mice and the animals were exposed either 95% oxygen/5% CO2 (carbogen) or medical air and the tumor bioluminescence imaged by IVIS 30 minutes post injection. Increasing oxygen breathing decreases reporter signal (FIG. 14B). These animals were then treated with either papaverine or vehicle (FIG. 14C) or SMV32 or vehicle (FIG. 14E). We see significant decrease in signal 2 and 6 hours following papaverine or SMV32, but no change in signal from vehicle. Control tumors bearing a non-hypoxia responsive luciferase (CMV luciferase) did not show any change in response to delivery of papaverine to the animal (FIG. 14D).

The compositions and methods of the appended claims are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only certain representative materials and method steps disclosed herein are specifically described, other combinations of the materials and method steps also are intended to fall within the scope of the appended claims, even if not specifically recited. Thus, a combination of steps, elements, components, or constituents may be explicitly mentioned herein; however, other combinations of steps, elements, components, and constituents are included, even though not explicitly stated. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed. As used in this disclosure and in the appended claims, the singular forms “a”, “an”, “the”, include plural referents unless the context clearly dictates otherwise.

1. A compound represented by a structure having the Formula I: 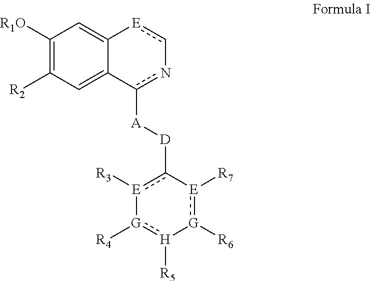
wherein
A and D are independently present or absent and are independently selected from CR′R″, NR′, and O, wherein R′ and R″ are independently for each occurrence selected from hydrogen, hydroxyl, halogen, amine, alkylamine, C₁-C₆ alkyl, C₁-C₆ alkyl halide, C₁-C₆ alkoxy, or R′ and R″ combine together with the atom to which they are attached form a carbonyl group;
E, G, and H are independently selected from C, N, O, and S;
R¹ and R² are independently selected from hydrogen, C₁-C₆ alkyl, C₁-C₆ alkyl halide, C₁-C₆ alkoxy, and C₁-C₆ alkylamine;
R³ to R⁷ are independently selected from hydrogen, hydroxyl, C₁-C₆ alkyl, C₁-C₆ alkyl halide, C₁-C₆ alkoxy, and C₁-C₆ alkylamine or R³ and R⁴ or R⁴ and R⁵ or R⁵ and R⁶ or R⁶ and R⁷ combine together with the atoms to which they are attached form a C₅-C₈ aryl or heteroaryl, or C₅-C₈ cycloalkenyl or heterocycloalkenyl; wherein R³ to R⁷ are independently unsubstituted or substituted with hydroxyl, halogen, C₁-C₃ alkyl, C₁-C₃ alkenyl, or C₁-C₃ alkyl halide; and
----- represents a bond and is independently for each occurrence absent or present,
wherein when A is CH₂ and D is absent, then R¹, R², R⁴, and R⁵ are not simultaneously OMe or
when A is CH₂, D is absent, R¹ and R² are OMe, then R⁴ and R⁵ do not combine to form and unsubstituted aryl.

2. The compound of claim 1, represented by a structure having the Formula I′: 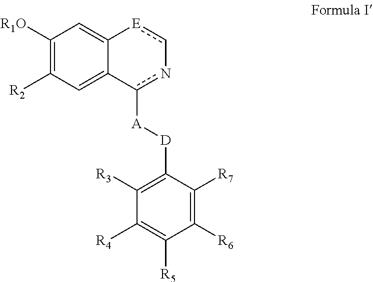
wherein
A and D are independently present or absent and are independently selected from CR′R″, NR′, and O, wherein R′ and R″ are independently for each occurrence selected from hydrogen, hydroxyl, halogen, amine, alkylamine, C₁-C₆ alkyl, C₁-C₆ alkyl halide, C₁-C₆ alkoxy, or R′ and R″ combine together with the atom to which they are attached form a carbonyl group;
E is selected from C and N;
R¹ and R² are independently selected from hydrogen, C₁-C₆ alkyl, C₁-C₆ alkyl halide, C₁-C₆ alkoxy, and C₁-C₆ alkylamine;
R³ to R⁷ are independently selected from hydrogen, hydroxyl, C₁-C₆ alkyl, C₁-C₆ alkyl halide, C₁-C₆ alkoxy, and C₁-C₆ alkylamine or R³ and R⁴ or R⁴ and R⁵ or R⁵ and R⁶ or R⁶ and R⁷ combine together with the atoms to which they are attached form a C₅-C₈ aryl or heteroaryl, or C₅-C₈ cycloalkenyl or heterocycloalkenyl; wherein R³ to R⁷ are independently unsubstituted or substituted with hydroxyl, halogen, C₁-C₃ alkyl, C₁-C₃ alkenyl, or C₁-C₃ alkyl halide; and
----- represents a bond and is independently for each occurrence absent or present,
wherein R⁴ and R⁵ or R⁵ and R⁶ are not simultaneously OMe, or
when A is CH₂ and D is absent, then R and R² are not simultaneously OMe.

3. The compound of claim 1, wherein the compound is represented by a structure having the Formula I-A to I-C: 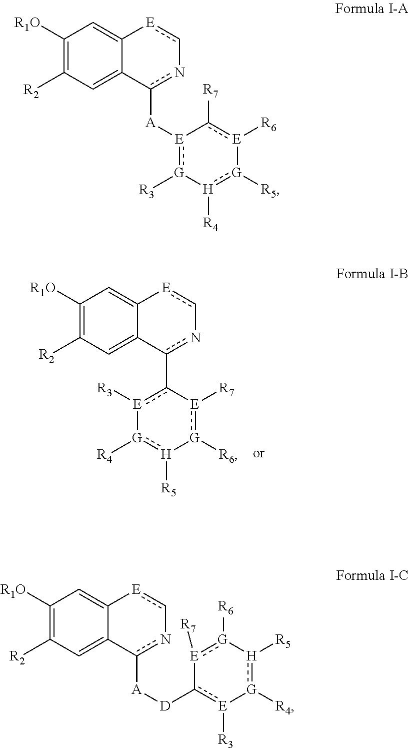
wherein
A is present in Formula I-A and
A and D are present in Formula I-C.

4. The compound of claim 1, wherein the compound is represented by a structure having the Formula I′-A to I′-C: 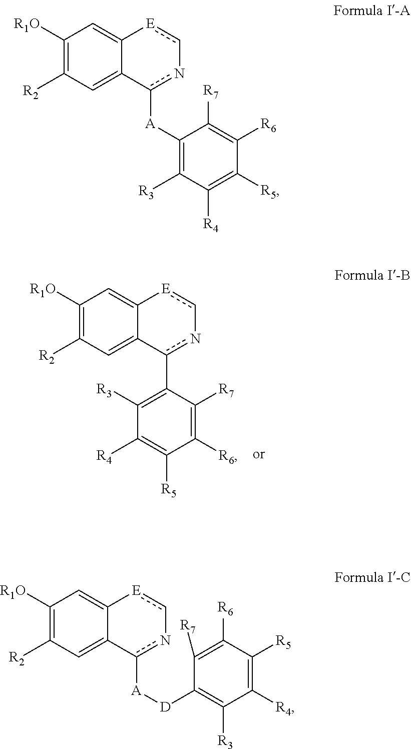
wherein
A is present in Formula I′-A and
A and D are present in Formula I′-C.

5. The compound of claim 1, wherein the compound is represented by a structure having the Formula I-A-1: 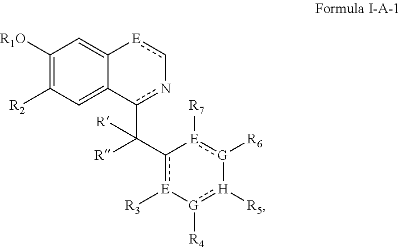
wherein
R′ and R″ are independently selected from hydrogen, hydroxyl, halogen, amine, alkylamine, C₁-C₆ alkyl, C₁-C₆ alkyl halide, C₁-C₆ alkoxy, or R′ and R″ combine together with the atom to which they are attached form a carbonyl;
R¹ is selected from hydrogen and C₁-C₆ alkyl;
R² is selected from hydrogen, hydroxyl, C₁-C₆ alkyl, and C₁-C₆ alkoxy; and
R³ to R⁷ are independently selected from hydrogen, hydroxyl, C₁-C₆ alkyl, C₁-C₆ alkyl halide, C₁-C₆ alkoxy, and C₁-C₆ alkylamine or R³ and R⁴ or R⁴ and R⁵ or R⁵ and R⁶ or R⁶ and R⁷ combine together with the atoms to which they are attached form a C₅-C₈ aryl or heteroaryl, or C₅-C₈ cycloalkenyl or heterocycloalkenyl,
wherein R³ to R⁷ are independently unsubstituted or substituted with hydroxyl, halogen, C₁-C₃ alkyl, C₁-C₃ alkenyl, or C₁-C₃ alkyl halide.

6. The compound of claim 1, wherein the compound is represented by a structure having the Formula I′-A-1: 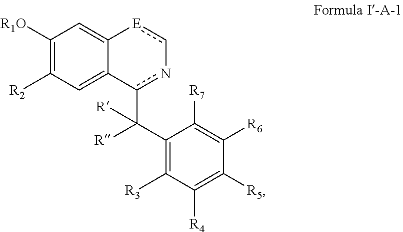
wherein
R′ and R″ are independently selected from hydrogen, hydroxyl, halogen, amine, alkylamine, C₁-C₆ alkyl, C₁-C₆ alkyl halide, C₁-C₆ alkoxy, or R′ and R″ combine together with the atom to which they are attached form a carbonyl;
R¹ is selected from hydrogen and C₁-C₆ alkyl;
R² is selected from hydrogen, hydroxyl, C₁-C₆ alkyl, and C₁-C₆ alkoxy; and
R³ to R⁷ are independently selected from hydrogen, hydroxyl, C₁-C₆ alkyl, C₁-C₆ alkyl halide, C₁-C₆ alkoxy, and C₁-C₆ alkylamine or R³ and R⁴ or R⁴ and R⁵ or R⁵ and R⁶ or R⁶ and R⁷ combine together with the atoms to which they are attached form a C₅-C₈ aryl or heteroaryl, or C₅-C₈ cycloalkenyl or heterocycloalkenyl,
wherein R³ to R⁷ are independently unsubstituted or substituted with hydroxyl, halogen, C₁-C₃ alkyl, C₁-C₃ alkenyl, or C₁-C₃ alkyl halide.

7. The compound of claim 1, wherein the compound is represented by a structure having the Formula I-B-1: 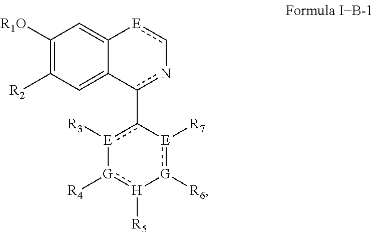
wherein
R¹ is selected from hydrogen and C₁-C₆ alkyl;
R² is selected from hydrogen, hydroxyl, C₁-C₆ alkyl, and C₁-C₆ alkoxy; and
R³ to R⁷ are independently selected from hydrogen, hydroxyl, C₁-C₆ alkyl, C₁-C₆ alkyl halide, C₁-C₆ alkoxy, and C₁-C₆ alkylamine or R³ and R⁴ or R⁴ and R⁵ or R⁵ and R⁶ or R⁶ and R⁷ combine together with the atoms to which they are attached form a C₅-C₈ aryl or heteroaryl, or C₅-C₈ cycloalkenyl or heterocycloalkenyl,
wherein R³ to R⁷ are independently unsubstituted or substituted with hydroxyl, halogen, C₁-C₃ alkyl, C₁-C₃ alkenyl, or C₁-C₃ alkyl halide.

8. The compound of claim 1, wherein the compound is represented by a structure having the Formula I′-B-1: 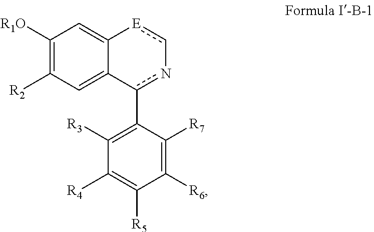
wherein
R¹ is selected from hydrogen and C₁-C₆ alkyl;
R² is selected from hydrogen, hydroxyl, C₁-C₆ alkyl, and C₁-C₆ alkoxy; and
R³ to R⁷ are independently selected from hydrogen, hydroxyl, C₁-C₆ alkyl, C₁-C₆ alkyl halide, C₁-C₆ alkoxy, and C₁-C₆ alkylamine or R³ and R⁴ or R⁴ and R⁵ or R⁵ and R⁶ or R⁶ and R⁷ combine together with the atoms to which they are attached form a C₅-C₈ aryl or heteroaryl, or C₅-C₈ cycloalkenyl or heterocycloalkenyl,
wherein R³ to R⁷ are independently unsubstituted or substituted with hydroxyl, halogen, C₁-C₃ alkyl, C₁-C₃ alkenyl, or C₁-C₃ alkyl halide.

9. The compound of claim 1, wherein the compound is represented by a structure having the Formula I-C-1: 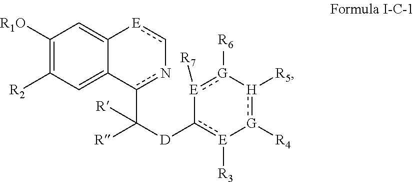
wherein
D is selected from CR′R″, NR′, and O,
R′ and R″ are independently selected from hydrogen, hydroxyl, halogen, amine, alkylamine, C₁-C₆ alkyl, C₁-C₆ alkyl halide, C₁-C₆ alkoxy, or R′ and R″ combine together with the atom to which they are attached form a carbonyl;
R¹ is selected from hydrogen and C₁-C₆ alkyl;
R² is selected from hydrogen, hydroxyl, C₁-C₆ alkyl, and C₁-C₆ alkoxy; and
R³ to R⁷ are independently selected from hydrogen, hydroxyl, C₁-C₆ alkyl, C₁-C₆ alkyl halide, C₁-C₆ alkoxy, and C₁-C₆ alkylamine or R³ and R⁴ or R⁴ and R⁵ or R⁵ and R⁶ or R⁶ and R⁷ combine together with the atoms to which they are attached form a C₅-C₈ aryl or heteroaryl, or C₅-C₈ cycloalkenyl or heterocycloalkenyl,
wherein R³ to R⁷ are independently unsubstituted or substituted with hydroxyl, halogen, C₁-C₃ alkyl, C₁-C₃ alkenyl, or C₁-C₃ alkyl halide.

10. The compound of claim 1, wherein the compound is represented by a structure having the Formula I′-C-1: 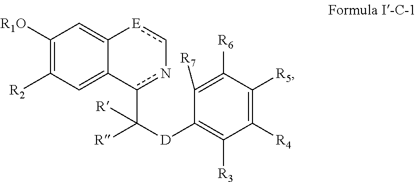
wherein
D is selected from CR′R″, NR′, and O,
R′ and R″ are independently selected from hydrogen, hydroxyl, halogen, amine, alkylamine, C₁-C₆ alkyl, C₁-C₆ alkyl halide, C₁-C₆ alkoxy, or R′ and R″ combine together with the atom to which they are attached form a carbonyl;
R¹ is selected from hydrogen and C₁-C₆ alkyl;
R² is selected from hydrogen, hydroxyl, C₁-C₆ alkyl, and C₁-C₆ alkoxy; and
R³ to R⁷ are independently selected from hydrogen, hydroxyl, C₁-C₆ alkyl, C₁-C₆ alkyl halide, C₁-C₆ alkoxy, and C₁-C₆ alkylamine or R³ and R⁴ or R⁴ and R⁵ or R⁵ and R⁶ or R⁶ and R⁷ combine together with the atoms to which they are attached form a C₅-C₈ aryl or heteroaryl, or C₅-C₈ cycloalkenyl or heterocycloalkenyl,
wherein R³ to R⁷ are independently unsubstituted or substituted with hydroxyl, halogen, C₁-C₃ alkyl, C₁-C₃ alkenyl, or C₁-C₃ alkyl halide.

11. The compound of claim 1, wherein A is selected from CR′R″ and O.

12. The compound of claim 1, wherein D is selected from CR′R″ and O.

13. The compound of claim 1, wherein A and D are both CR′R″.

14. The compound of claim 1, wherein A is CR′R″ and D is O.

15. The compound of claim 1, wherein R′ and R″ are independently for each occurrence selected from hydrogen, hydroxyl, C₁-C₆ alkyl, or R′ and R″ combine together with the atom to which they are attached form a carbonyl.

16. The compound of claim 1, wherein R′ and R″ are hydrogen.

17. The compound of claim 1, wherein R′ is hydrogen and at least one occurrence of R″ is hydroxyl.

18. The compound of claim 1, wherein at least one occurrence of R′ and R″ combine together with the atom to which they are attached form a carbonyl.

19. The compound of claim 1, wherein R¹ is selected from a C₁-C₆ alkyl.

20. The compound of claim 1, wherein R¹ is selected from a C₁-C₂ alkyl.

21. The compound of claim 1, wherein R² is independently selected from hydrogen or a C₁-C₆ alkoxy.

22. The compound of claim 1, wherein R² is selected from a C₁-C₂ alkoxy.

23. The compound of claim 1, wherein R² is hydrogen.

24. The compound of claim 1, wherein R³, R⁴, R⁵, R⁶, and R⁷ are independently selected from hydrogen, hydroxyl, C₁-C₆ alkyl, C₁-C₆ alkyl halide, C₁-C₆ alkoxy, or R³ and R⁴ or R⁴ and R⁵ combine together with the atoms to which they are attached form a C₅-C₈ aryl or heteroaryl, or C₅-C₈ cycloalkenyl or heterocycloalkenyl, wherein R³ to R⁷ are independently unsubstituted or substituted with hydroxyl, halogen, C₁-C₃ alkyl, C₁-C₃ alkenyl, or C₁-C₃ alkyl halide.

25. The compound of claim 1, wherein R³ and R⁷ are hydrogen.

26. The compound of claim 1, wherein R⁴, R⁵, and R⁶ are independently selected from hydrogen, hydroxyl, C₁-C₆ alkyl, C₁-C₆ alkyl halide, or C₁-C₆ alkoxy.

27. The compound of claim 1, wherein R³ and R⁴ or R⁴ and R⁵ combine together with the atoms to which they are attached form a C₆ aryl, a C₆ heteroaryl, or a C₅ heterocycloalkenyl.

28. The compound of claim 1, wherein the compound is represented by a structure below:

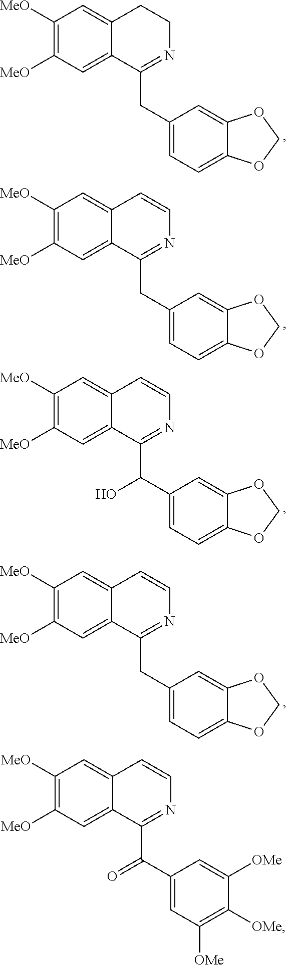

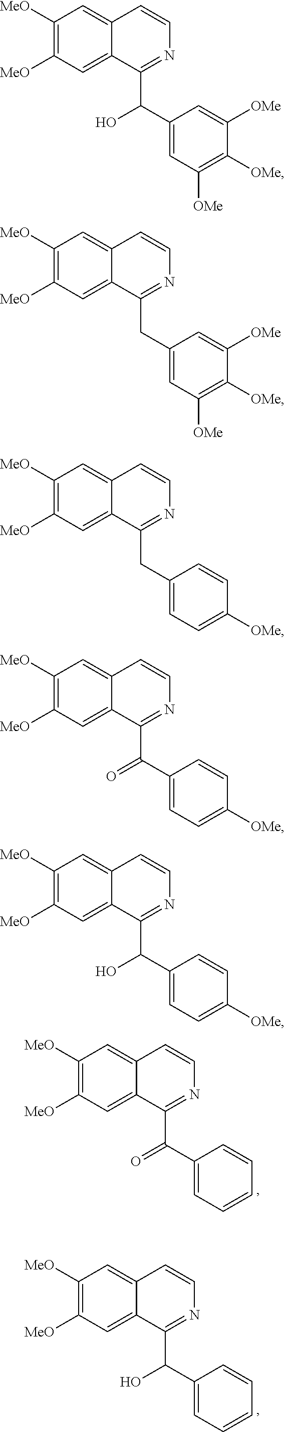

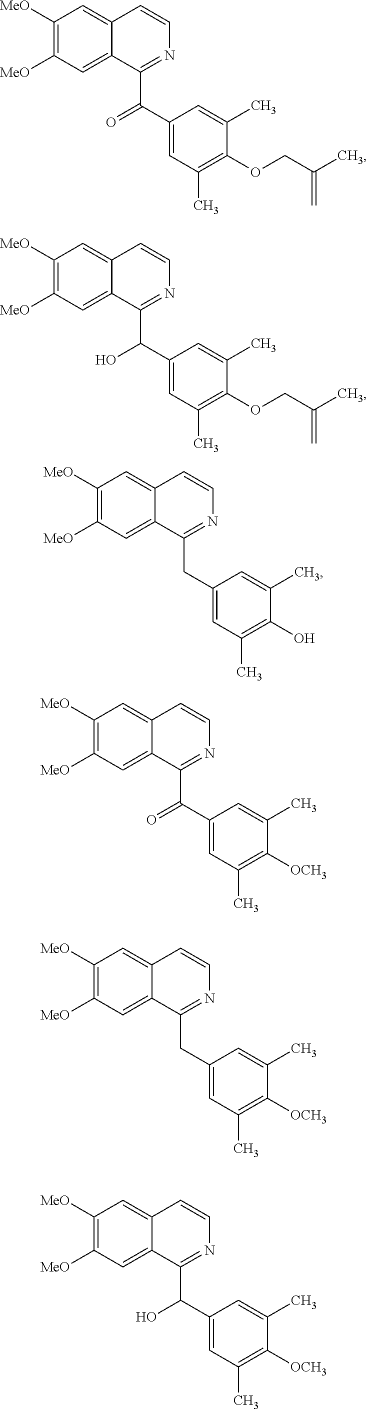

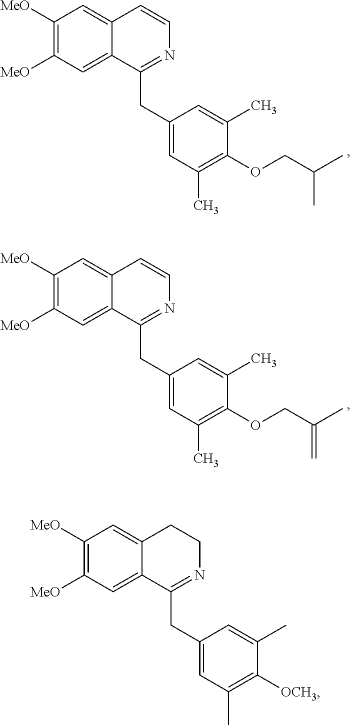 [SMILES: COc1cc2ccnc(Cc3cc(C)c(OCC(C)C)c(C)c3)c2cc1OC ; C=C(C)COc1c(C)cc(Cc2nccc3cc(OC)c(OC)cc23)cc1C ; COc1cc2c(cc1OC)C(Cc1cc(C)c(OC)c(C)c1)=NCC2]

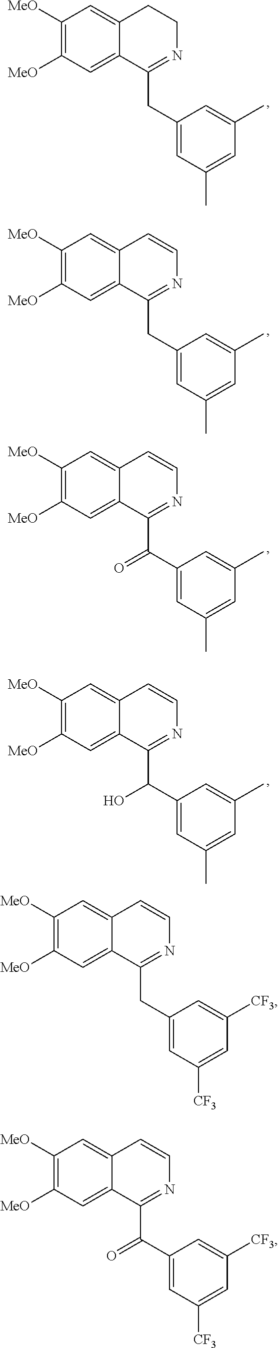

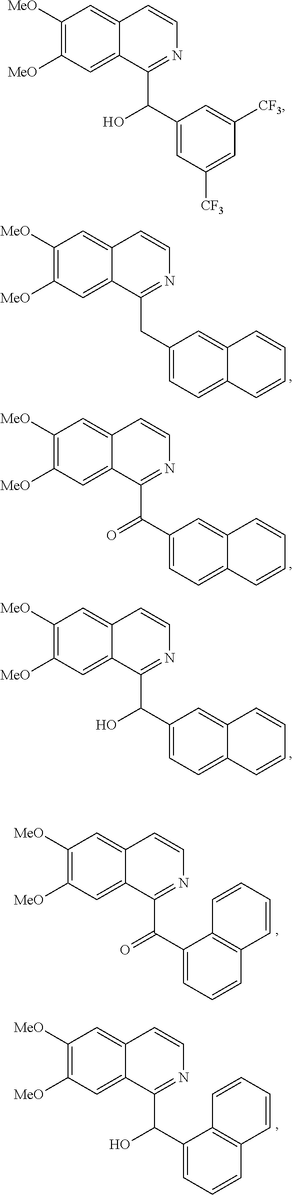 [SMILES: COc1cc2ccnc(C(O)c3cc(C(F)(F)F)cc(C(F)(F)F)c3)c2cc1OC ; COc1cc2ccnc(Cc3ccc4ccccc4c3)c2cc1OC ; COc1cc2ccnc(C(=O)c3ccc4ccccc4c3)c2cc1OC ; COc1cc2ccnc(C(O)c3ccc4ccccc4c3)c2cc1OC ; COc1cc2ccnc(C(=O)c3cccc4ccccc34)c2cc1OC ; COc1cc2ccnc(C(O)c3cccc4ccccc34)c2cc1OC]

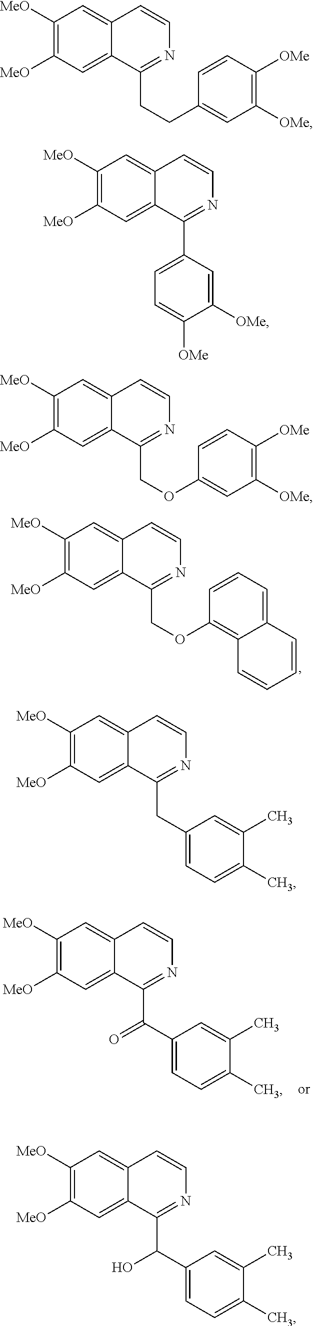

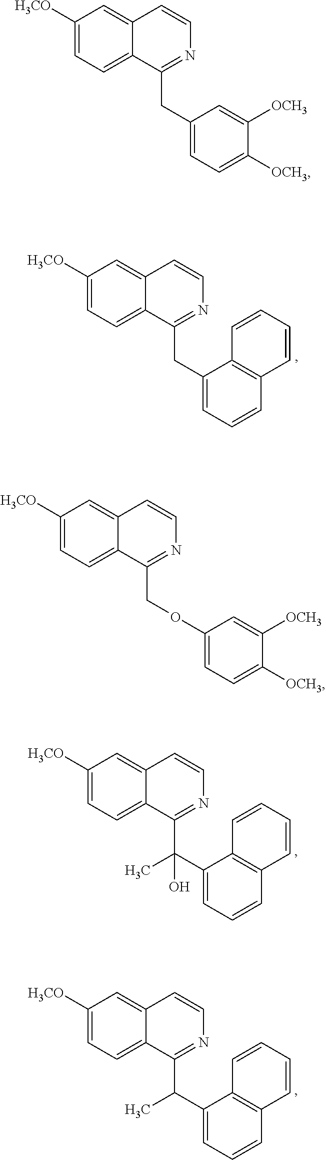 [SMILES: COc1ccc2c(Cc3ccc(OC)c(OC)c3)nccc2c1 ; COc1ccc2c(Cc3cccc4ccccc34)nccc2c1 ; COc1ccc2c(COc3ccc(OC)c(OC)c3)nccc2c1 ; COc1ccc2c(C(C)(O)c3cccc4ccccc34)nccc2c1 ; COc1ccc2c(C(C)c3cccc4ccccc34)nccc2c1]

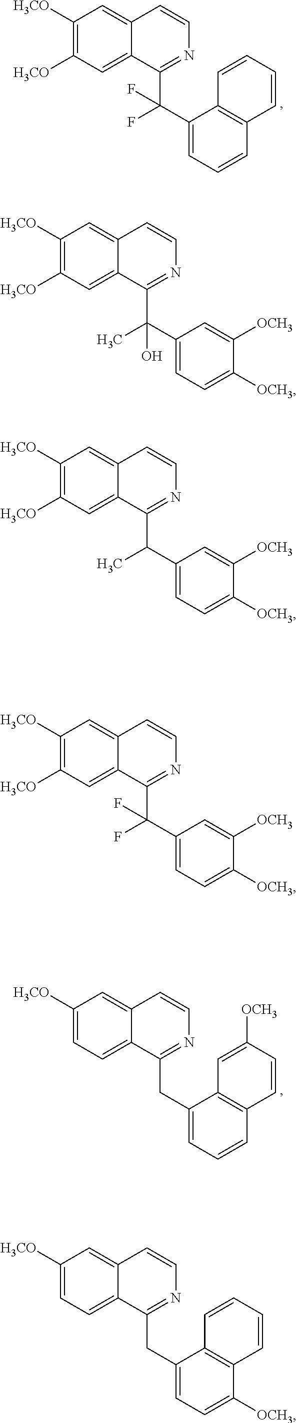 [SMILES: COc1cc2ccnc(C(F)(F)c3cccc4ccccc34)c2cc1OC ; COc1ccc(C(C)(O)c2nccc3cc(OC)c(OC)cc23)cc1OC ; COc1ccc(C(C)c2nccc3cc(OC)c(OC)cc23)cc1OC ; COc1ccc(C(F)(F)c2nccc3cc(OC)c(OC)cc23)cc1OC ; COc1ccc2c(Cc3cccc4ccc(OC)cc34)nccc2c1 ; COc1ccc2c(Cc3ccc(OC)c4ccccc34)nccc2c1]

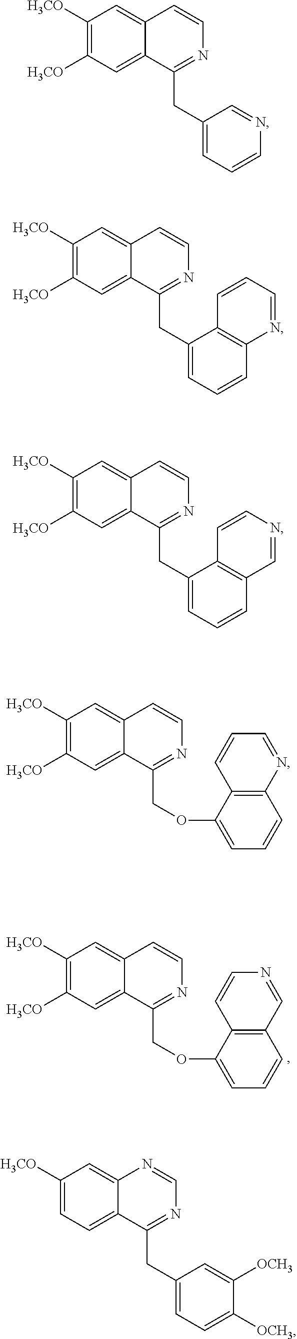 [SMILES: COc1cc2ccnc(Cc3cccnc3)c2cc1OC ; COc1cc2ccnc(Cc3cccc4ncccc34)c2cc1OC ; COc1cc2ccnc(Cc3cccc4cnccc34)c2cc1OC ; COc1cc2ccnc(COc3cccc4ncccc34)c2cc1OC ; COc1cc2ccnc(COc3cccc4cnccc34)c2cc1OC ; COc1ccc2c(Cc3ccc(OC)c(OC)c3)ncnc2c1]

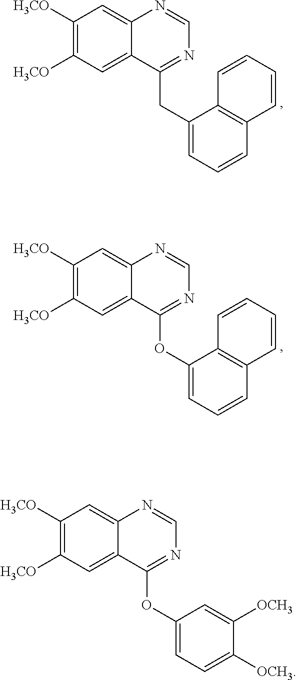 [SMILES: COc1cc2ncnc(Cc3cccc4ccccc34)c2cc1OC ; COc1cc2ncnc(Oc3cccc4ccccc34)c2cc1OC ; COc1ccc(Oc2ncnc3cc(OC)c(OC)cc23)cc1OC]

29. A pharmaceutical composition or formulation comprising a compound according to claim 1.

30. A method for inhibiting mitochondrial oxygen consumption in a cancerous tissue within a subject, the method comprising administering to the subject a pharmaceutical composition according to claim 29.

31. (canceled)

32. A method for treating a cancerous tissue in a subject comprising:
(a) administering to the subject a pharmaceutical composition comprising a compound having a structure according to Formula II for causing inhibition of mitochondrial oxygen consumption in the cancerous tissue 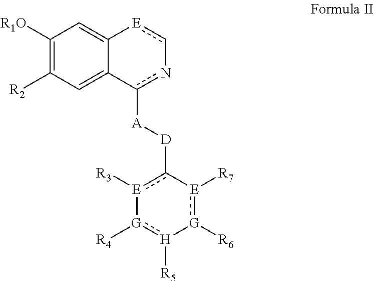
wherein
A and D are independently present or absent and are independently selected from CR′R″, NR′, and O, wherein R′ and R″ are independently for each occurrence selected from hydrogen, hydroxyl, halogen, amine, alkylamine, C₁-C₆ alkyl, C₁-C₆ alkyl halide, C₁-C₆ alkoxy, or R′ and R″ combine together with the atom to which they are attached form a carbonyl group;
E, G, and H can be independently selected from C, N′, O, and S;
R¹ and R² are independently selected from hydrogen, C₁-C₆ alkyl, C₁-C₆ alkyl halide, C₁-C₆ alkoxy, and C₁-C₆ alkylamine;
R³ to R⁷ are independently selected from hydrogen, hydroxyl, C₁-C₆ alkyl, C₁-C₆ alkyl halide, C₁-C₆ alkoxy, and C₁-C₆ alkylamine or R³ and R⁴ or R⁴ and R⁵ or R⁵ and R⁶ or R⁶ and R⁷ combine together with the atoms to which they are attached form a C₅-C₈ aryl or heteroaryl, or C₅-C₈ cycloalkenyl or heterocycloalkenyl; wherein R³ to R⁷ are independently unsubstituted or substituted with hydroxyl, halogen, C₁-C₃ alkyl, C₁-C₃ alkenyl, or C₁-C₃ alkyl halide; and
----- represents a bond and is independently for each occurrence absent or present; and
(b) irradiating the cancerous tissue with an ionizing radiation for an effective period to treat the cancerous tissue.

33-43. (canceled)
